(19) United States
(12) Patent Application Publication
(10) Pub. No.: US 2010/0267765 A1
(43) Pub. Date: Oct. 21, 2010

(54) Pharmaceutical Compositions and Methods for CCR5 Antagonists

(76) Inventors: Stephen John Felstead, Kent (GB); Andrew Lee Hopkins, Kent (GB); Howard Bernard Mayer, New York, NY (US); Mary McHale, Kent (GB)

Correspondence Address: PFIZER INC.;PATENT DEPARTMENT, Bld 114 M/S 9114, EASTERN POINT ROAD, GROTON, CT, 06340, US

(21) Appl. No.: 12/526,795

(22) PCT Filed: Feb. 8, 2008
(86) PCT No.: PCT/IB08/00361

§ 371 Date: May 5, 2010

Related U.S. Application Data
(60) Provisional application No. 60/890,096, filed on Feb. 15, 2007.

(51) Int. Cl. A61K 31/46; C07D 451/02; A61P 9/10; A61P 3/06; A61P 3/10; A61P 3/04

(52) U.S. Cl. 514/304; 546/125

(57) ABSTRACT

The present invention relates to a CCR5 antagonist compound for elevating high density lipoprotein (HDL) particles in a patient, improving plasma lipid profile in a patient or reducing triglycerides in a patient. The invention also relates to a pharmaceutical composition comprising a CCR5 antagonist compound, an HMG-CoA reductase inhibitor compound and a pharmaceutically acceptable carrier. The invention also relates to a pharmaceutical composition comprising a CCR5 antagonist compound, a cholesteryl ester transfer protein (CETP) inhibitor compound and a pharmaceutically acceptable carrier.

### FIELD OF THE INVENTION

This invention relates to the use of a CCR5 antagonist to elevate certain plasma lipid levels, including high density lipoprotein (HDL)-cholesterol in patients in need thereof, such as HIV infected patients and patients with atherosclerosis or lipid dysfunctions. The invention also relates to the use of a CCR5 antagonist to lower certain plasma lipid levels, including triglycerides in patients in need thereof such as patients with hypertriglyceridemia, atherosclerosis, dyslipidemia, hypercholesterolemia, cardiovascular diseases. The invention also relates to the combination of a CCR5 antagonist and other pharmaceutical agents, such as HMG-CoA reductase inhibitors, especially atorvastatin, to elevate certain plasma lipid levels, including high density lipoprotein (HDL)-cholesterol, and to lower other plasma lipid levels, such as low density lipoprotein (LDL)-cholesterol and triglycerides, in patients in need thereof, and accordingly to treat diseases which are affected by low levels of HDL cholesterol and/or high levels of LDL-cholesterol and triglycerides, such as atherosclerosis, dyslipidemia, hypercholesterolemia, hypertriglyceridemia, cardiovascular diseases and related diseases such as diabetes. The present invention also relates to the further combination of a CCR5 antagonist, an HMG-CoA reductase inhibitor and a CETP inhibitor for improving the plasma lipid profile of a patient in need thereof, as described above. The present invention also relates to pharmaceutical compositions and kits that comprise a CCR5 antagonist and a second or third therapeutic agent.

### BACKGROUND OF THE INVENTION

Atherosclerosis and its associated coronary artery disease (CAD) is the leading cause of mortality in the industrialized world. Despite attempts to modify secondary risk factors (e.g., smoking, obesity, lack of exercise) and treatment of dyslipidemia with dietary modification and drug therapy, coronary heart disease (CHD) remains the most common cause of death in the U.S., where cardiovascular disease accounts for 44% of all deaths, with 53% of these associated with atherosclerotic coronary heart disease.

The pathological sequence leading to atherosclerosis and coronary heart disease is well known. The earliest stage in this sequence is the formation of “fatty streaks” in the carotid, coronary and cerebral arteries and in the aorta. These lesions are yellow in color due to the presence of lipid deposits found principally within smooth-muscle cells and in macrophages of the intima layer of the arteries and aorta. Further, it is postulated that most of the cholesterol found within the fatty streaks, in turn, give rise to development of “fibrous plaques,” which consist of accumulated intimal smooth muscle cells laden with lipid and are surrounded by extra-cellular lipid, collagen, elastin and proteoglycans. The cells plus matrix form a fibrous cap that covers a deeper deposit of cell debris and more extra-cellular lipid. The lipid is primarily free and esterified cholesterol. The fibrous plaque forms slowly, and is likely in time to become calcified and necrotic, advancing to a “complicated lesion,” which accounts for arterial occlusion and tendency toward mural thrombosis and arterial muscle spasm that characterize advanced atherosclerosis.

Risk for development of atherosclerosis and related cardiovascular disease has been shown to be strongly correlated with certain plasma lipid levels. In recent years, leaders of the medical profession have placed renewed emphasis on lowering plasma cholesterol levels, and low density lipoprotein (LDL)-cholesterol, in particular. The upper limits of “normal” are now known to be significantly lower than heretofore appreciated. As a result, large segments of Western populations are now realized to be at particularly high risk. Such independent risk factors include glucose intolerance, left ventricular hypertrophy, hypertension, and being of the male sex. Cardiovascular disease is especially prevalent among diabetic subjects, at least in part because of the existence of multiple independent risk factors in this population. Successful treatment of hyperlipidemia in the general population, and in diabetic subjects in particular, is therefore of exceptional medical importance.

While elevated LDL-cholesterol may be the most recognized form of dyslipidemia, it is by no means the only significant lipid associated contributor to CHD. Low HDL-C is also a known risk factor for CHD (D. J. Gordon et al., “High-density Lipoprotein Cholesterol and Cardiovascular Disease,” Circulation (1989) 79: 8-15). High LDL-cholesterol and triglyceride levels are positively correlated, while high levels of HDL-cholesterol are negatively correlated with the risk for developing cardiovascular diseases. Thus, dyslipidemia is not a unitary risk profile for CHD but may be comprised of one or more lipid aberrations.

No wholly satisfactory lipid-modulating therapies exist. Niacin can significantly increase HDL-cholesterol, but has serious toleration issues, which reduce compliance. Fibrates and the HMG-CoA reductase inhibitors lower LDL-cholesterol but raise HDL-cholesterol only modestly (on average less than about 20%). As a result, there is a significant unmet medical need for a well-tolerated agent, which can lower plasma LDL levels and/or elevate plasma HDL levels (i.e., improving the patient's plasma lipid profile), thereby reversing or slowing the progression of certain diseases.

Thus, although there are therapies available, there is a continuing need and a continuing search for alternative therapies for the treatment of diseases which are affected by low levels of HDL cholesterol and/or high levels of LDL-cholesterol and triglycerides, such as atherosclerosis, plaque formation, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia, myocardial infarction, metabolic syndrome, obesity and diabetes.

Maraviroc, a CCR5 antagonist, chemical name, (N-{(1S)-3-[3-isopropyl-5-methyl-4H-1,2,4-triazole-4-yl]-exo-8-azabicyclo[3.2.1]oct-8-yl}-1-phenylpropyl)-4,4-difluorocyclohexanecarboxamide), which is disclosed in WO 01/90106 (incorporated herein by reference), is a chemokine receptor antagonist which inhibits entry of HIV through the CCR5 co-receptor. Methods to make Maraviroc are disclosed in WO 01/90106. Maraviroc has recently been launched as Celsentri™ or Selzentry™ for the treatment of patients infected with CCR5 tropic HIV-1.

Other CCR5 antagonists have been described in many literature and patent references, including WO 2005/033107; WO 03/084954; and others referenced below.

WO 0004926A2 and WO 0004926A3 disclose conjugates for treating inflammatory disorders and associated tissue damage.

An editorial in Arterioscler. Thromb. Vasc. Biol. 2005; 25:2448-2450, describes targeting chemokine receptors in atherosclerosis and HIV infection.

Studies of humans with the CCR5 delta 32 deletion, which is associated with reduced cell-surface expression of CCR5 receptors, have shown that these individuals have a lower incidence of early myocardial infarction (Gonzalez P, et al. Genes Immun 2001; 2:191-195) and a lower risk of severe coronary artery disease (Szalai C, et al. Atherosclerosis 2001; 158:233-239). Furthermore, mice at risk for atherosclerosis that were treated with a CCR5 antagonist showed delayed progression of atherosclerosis compared to untreated mice (Veillard N R, et al. Circ Res 2004; 94:253-261, van Wanrooij E J, et al. Arterioscler Thromb Vasc Biol 2005; 25:2642-2647). However, the relevance of these mouse model data to CCR5 antagonism in humans is unclear.

### SUMMARY OF THE INVENTION

The present invention provides the following uses for a CCR5 antagonist compound: use of a CCR5 antagonist compound for the preparation of a medicament for elevating high density lipoprotein (HDL) particles in a patient; use of a CCR5 antagonist compound for the preparation of a medicament for improving plasma lipid profile in a patient; and use of a CCR5 antagonist compound for the preparation of a medicament for reducing triglycerides in a patient. The present invention also provides such uses for a CCR5 antagonist compound when administered to a patient in need thereof.

The present invention also provides the following uses for a CCR5 antagonist compound: use of a CCR5 antagonist compound for the preparation of a medicament for reducing total cholesterol levels in a patient and use of a CCR5 antagonist compound for the preparation of a medicament for reducing low density lipoprotein (LDL) particles in a patient. The present invention also provides such uses for a CCR5 antagonist compound when administered to a patient in need thereof.

In one aspect, the present invention provides the above uses wherein the patient is infected with HIV. The present invention provides the above uses wherein the patient is infected with a CXCR4 virus using HIV viral population. In one embodiment, the present invention provides the above uses wherein the viral population of the HIV patient contains more than 10% CXCR4 virus. The following additional embodiments of the present invention are also provided: wherein the viral population of the HIV patient contains more than 20% CXCR4 virus; wherein the viral population of the HIV patient contains more than 30% CXCR4 virus; wherein the viral population of the HIV patient contains more than 40% CXCR4 virus; and wherein the viral population of the HIV patient contains more than 50% CXCR4 virus.

In one aspect, the present invention provides the above uses wherein the CCR5 antagonist is selected from maraviroc, vicrviroc, NCB-9471, PRO-140, CCR5 mAb004, 8-[4-(2-butoxyethoxy)phenyl]-1-isobutyl-N-[4-[[(1-propyl-1H-imadazol-5-yl)methyl]sulphinyl]phenyl]-1,2,3,4-tetrahydro-1-benzacocine-5-carboxamide, methyl1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carboxylate, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, and N-{(1S)-3-[3-endo-(5-isobutyryl-2-methyl-4,5,6,7-tetrahydro-1H-imidazo[4,5-c]pyridin-yl)-8-azabicyclo[3.2.1]oct-8-yl]-1-(3-fluorophenyl)propyl}acetamide), and/or a pharmaceutically acceptable salt and/or solvate thereof. More specifically, the present invention provides the above uses wherein the CCR5 antagonist is maraviroc or a pharmaceutically acceptable salt or solvate thereof. Most preferably, the CCR5 antagonist compound used in the aspects of the invention disclosed herein is maraviroc in the free base form.

In addition, the present invention provides the above uses wherein the HIV patient is taking at least a protease inhibitor or a NRTI. The present invention also provides the above uses wherein the patient is diagnosed with a disease which is affected by low levels of HDL cholesterol and/or high levels of LDL-cholesterol and triglycerides, wherein the disease is selected from atherosclerosis, plaque formation, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia, myocardial infarction, metabolic syndrome, obesity and diabetes.

In another aspect of the invention, there is provided a CCR5 antagonist compound for the hereinabove-mentioned uses in a patient. In another aspect, the patient is infected with HIV as described in the above-mentioned aspects of the invention. Preferably, the CCR5 antagonist compound is selected from one of the CCR5 antagonist compounds disclosed herein. More preferably, the CCR5 antagonist compound is maraviroc or a pharmaceutically acceptable salt thereof. Most preferably, the CCR5 antagonist compound used in the aspects of the invention disclosed herein is maraviroc in the free base form.

In another aspect, the present invention provides a pharmaceutical composition comprising: a) a CCR5 antagonist compound; b) an HMG-CoA reductase inhibitor compound; and c) a pharmaceutically acceptable carrier. More particularly, the present invention provides such compositions wherein the HMG-CoA reductase inhibitor compound is selected from lovastatin, simvastatin, pravastatin, fluvastatin, atorvastatin, rivastatin, rosuvastatin and pitavastatin or a pharmaceutically acceptable salt or solvate thereof. More particularly, the present invention provides such compositions wherein the HMG-CoA reductase inhibitor compound is atorvastatin or a pharmaceutically acceptable salt or solvate thereof. Even more specifically, the present invention provides such compositions wherein the HMG-CoA reductase inhibitor compound is atorvastatin or a pharmaceutically acceptable salt thereof.

Also, in one aspect, the present invention provides such compositions wherein the CCR5 antagonist is as defined above. More specifically, the present invention provides such compositions wherein the CCR5 antagonist is maraviroc or a pharmaceutically acceptable salt or solvate thereof. In yet another aspect, the present invention provides such compositions which further comprise a CETP inhibitor compound, preferably cis-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide; or (2R)-3-{[3-(4-Chloro-3-ethyl-phenoxy)-phenyl]-[[3-(1,1,2,2-tetrafluoro-ethoxy)-phenyl]-methyl]-amino}-1,1,1-trifluoro-2-propanol or a pharmaceutically acceptable salt thereof.

In another aspect, the present invention provides a pharmaceutical composition comprising: a CCR5 antagonist compound; b) a CETP inhibitor compound; and c) a pharmaceutically acceptable carrier. The present invention provides such compositions wherein the CETP inhibitor compound is cis-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide; or (2R)-3-{[3-(4-Chloro-3-ethyl-phenoxy)-phenyl]-[[3-(1,1,2,2-tetrafluoro-ethoxy)-phenyl]-methyl]-amino}-1,1,1-trifluoro-2-propanol or a pharmaceutically acceptable salt thereof.

In yet another aspect, the present invention provides such compositions which further comprise a cholesterol absorption inhibitor compound.

Also, in one aspect, the present invention provides such compositions wherein the CCR5 antagonist compound is as defined above. More specifically, the present invention provides such compositions wherein the CCR5 antagonist is maraviroc or a pharmaceutically acceptable salt or solvate thereof.

Particularly, the present invention provides a composition as described hereinabove for the treatment of atherosclerosis, plaque formation, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia, myocardial infarction, metabolic syndrome, obesity or diabetes.

In another aspect, the present invention provides uses of the compositions of the present invention for improving plasma lipid profile in a mammal in need thereof.

In another aspect, the present invention provides kits for elevating high density lipoprotein (HDL) particles in a mammal in need thereof which comprises: a) a CCR5 antagonist compound and a pharmaceutically acceptable carrier, vehicle or diluent in a first unit dosage form; b) an HMG-CoA reductase inhibitor compound and a pharmaceutically acceptable carrier, vehicle or diluent in a second unit dosage form; and c) a means for containing the first and second unit dosage forms. More specifically, the present invention provides such kits wherein the HMG-CoA reductase inhibitor compound is selected from lovastatin, simvastatin, pravastatin, fluvastatin, atorvastatin, rivastatin, rosuvastatin and pitavastatin or a pharmaceutically acceptable salt or solvate thereof. More specifically, the present invention provides such kits wherein the HMG-CoA reductase inhibitor is atorvastatin or a pharmaceutically acceptable salt or solvate thereof. Even more specifically, the present invention provides such kits wherein the HMG-CoA reductase inhibitor is atorvastatin or a pharmaceutically acceptable salt thereof.

Also, in one aspect, the present invention provides such kits wherein the CCR5 antagonist is as defined above. More specifically, the present invention provides such kits wherein the CCR5 antagonist is maraviroc or a pharmaceutically acceptable salt or solvate thereof.

In another aspect, the present invention provides the following methods: a method for elevating high density lipoprotein (HDL) particles in a patient in need thereof which comprises the administration of a therapeutically effective amount of a CCR5 antagonist to the patient; a method for improving plasma lipid profile in a patient in need thereof which comprises the administration of a therapeutically effective amount of a CCR5 antagonist to the patient; and a method for reducing triglycerides in a patient in need thereof which comprises the administration of a therapeutically effective amount of a CCR5 antagonist to the patient.

In one aspect, the present invention provides the above methods wherein the patient is infected with HIV. The present invention provides the above methods wherein the patient is infected with a CXCR4 virus using HIV viral population. In one embodiment, the present invention provides the above methods wherein the viral population of the HIV patient contains more than 10% CXCR4 virus. The following additional embodiments of the present invention are also provided: wherein the viral population of the HIV patient contains more than 20% CXCR4 virus; wherein the viral population of the HIV patient contains more than 30% CXCR4 virus; wherein the viral population of the HIV patient contains more than 40% CXCR4 virus; and wherein the viral population of the HIV patient contains more than 50% CXCR4 virus.

In one aspect, the present invention provides the above methods wherein the CCR5 antagonist is selected from maraviroc, vicriviroc, NCB-9471, PRO-140, CCR5 mAb004, 8-[4-(2-butoxyethoxy)phenyl]-1-isobutyl-N-[4-[[(1-propyl-1H-imadazol-5-yl)methyl]sulphinyl]phenyl]-1,2,3,4-tetrahydro-1-benzacocine-5-carboxamide, methyl1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carboxylate, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, and N-{(1S)-3-[3-endo-(5-isobutyryl-2-methyl-4,5,6,7-tetrahydro-1H-imidazo[4,5-c]pyridin-1-yl)-8-azabicyclo[3.2.1]oct-8-yl]-1-(3-fluorophenyl)propyl}acetamide) and a pharmaceutically acceptable salt and solvate thereof. More specifically, the present invention provides the above methods wherein the CCR5 antagonist is maraviroc or a pharmaceutically acceptable salt or solvate thereof.

In addition, the present invention provides the above methods wherein the HIV patient is taking at least a protease inhibitor or a NRTI. The present invention also provides the above methods wherein the patient is diagnosed with a disease which is affected by low levels of HDL cholesterol and/or high levels of LDL-cholesterol and triglycerides, wherein the disease is selected from atherosclerosis, plaque formation, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia, myocardial infarction, metabolic syndrome, obesity and diabetes.

Preferably, the disease treated by the uses, methods and compositions of the invention is selected from atherosclerosis, dyslipidemia, hypercholesterolemia, hypertriglyceridemia, cardiovascular disease and diabetes, more preferably atherosclerosis and dyslipidemia. The invention includes the use as described herein of a CCR5 antagonist compound as described herein, wherein the patient is diagnosed with dyslipidemia or atherosclerosis associated with HIV and/or its treatment. The invention also includes a CCR5 antagonist compound as described herein, for use as described herein, wherein the patient is diagnosed with dyslipidemia or atherosclerosis associated with HIV and/or its treatment.

More particularly, the present invention provides such a method which further comprises administering a HMG-CoA reductase inhibitor compound, preferably selected from lovastatin, simvastatin, pravastatin, fluvastatin, atorvastatin, rivastatin, rosuvastatin and pitavastatin or a pharmaceutically acceptable salt thereof, more preferably atorvastatin or a pharmaceutically acceptable salt or solvate thereof, most preferably atorvastatin or a pharmaceutically acceptable salt thereof.

### BRIEF DESCRIPTION OF THE DRAWINGS

FIG. 1b shows the median maximum change from baseline in lipid parameters.

FIG. 2b shows the median maximum change from baseline in LDL cholesterol levels by baseline NCEP cholesterol category.

FIG. 3b shows the percentage of patients whose lipid values exceeded cutpoints for starting LDL-lowering therapy according to NCEP guidelines at one or more on-study assessments.

FIG. 4b shows the percentage of patients who exceeded cutpoints for starting LDL-lowering therapy according to NCEP guidelines at both Weeks 24 and 48.

FIG. 5b shows relative risk (and 95% confidence intervals) of having a CHD event within 10 years (Framingham equation) at a simulated smoking rate of 50%.

### DETAILED DESCRIPTION OF THE INVENTION

The present invention may be understood more readily by reference to the following detailed description of exemplary embodiments of the invention and the examples included therein.

Before the present compounds, compositions and methods are disclosed and described, it is to be understood that this invention is not limited to specific synthetic methods of making that may of course vary. It is also to be understood that the terminology used herein is for the purpose of describing particular embodiments only and is not intended to be limiting.

According to a first aspect of the invention there is provided the use of a CCR5 antagonist to increase HDL levels in a human patient.

In one embodiment of the invention, the patient is infected with human immunodeficiency virus (HIV).

In a further embodiment of the invention, the HIV patient is infected with a CXCR4 using viral population.

In a yet further embodiment of the invention, the HIV patient is taking at least one of a protease inhibitor or a nucleoside/nucleotide reverse transcriptase inhibitor (NRTI) as part of their HIV therapy.

The term “therapeutically effective amount” means an amount of a compound or combination of compounds that treats a disease; ameliorates, attenuates, or eliminates one or more symptoms of a particular disease; or prevents or delays the onset of one of more symptoms of a disease.

The term “patient” means animals, such as dogs, cats, cows, horses, sheep, geese, and humans. Particularly preferred patients are mammals, including humans of both sexes.

The term “pharmaceutically acceptable” means that the substance or composition must be compatible with the other ingredients of a formulation, and not deleterious to the patient.

The term “pharmaceutically acceptable salts” includes the salts of compounds that are, within the scope of sound medical judgment, suitable for use with patients without undue toxicity, irritation, allergic response, and the like, commensurate with a reasonable benefit/risk ratio, and effective for their intended use, as well as the zwitterionic forms, where possible, of the compounds.

Certain compounds of the present invention can exist in unsolvated form as well as solvated form including hydrated form (hydrate). In general, the solvated form including hydrated form (hydrate) is equivalent to the unsolvated form and is intended to be encompassed within the scope of the present invention.

The terms “treating”, “treat” or “treatment” include preventative (e.g., prophylactic) and palliative treatment.

CCR5 antagonists are antagonists of the chemotactic cytokine receptor type 5.

A human CD4 positive cell has both CCR5 and CXCR4 co-receptors on its surface, which it is thought HIV uses to gain entry to the cells. However different populations of the virus exist and can be classified according to the co-receptor (CCR5 or CXCR4) which they would normally use for cell entry. Hereinafter viral populations containing substantially CCR5 virus are classified as CCR5 tropic. Viral populations containing substantially CXCR4 virus are classified as CXCR4 tropic, viral populations with both CCR5 and CXCR4 virus are classified as mixed tropic, while a dual tropic virus can enter the CD4 cell via either the CCR5 or CXCR4 co-receptor. Herein a CXCR4 using viral population is classified as that containing some CXCR4 virus, preferably more than 2% CXCR4 virus, more preferably more than 5% CXCR4 virus, most preferably more than 10% CXCR4 virus.

An assay has therefore been developed to determine the tropism of the viral population that HIV patients are infected with, and accordingly provide appropriate treatment. In particular, CCR5 antagonists, such as maraviroc, are being developed for treatment of patients infected with a CCR5 tropic HIV viral population (rather than a CXCR4 using viral population).

The Phenosense™ (Trofile) assay (Monogram Biosciences, Califonia, USA) can be used to determine if an HIV patient is CCR5 tropic, and if so, maraviroc can then be administered. Maraviroc is not indicated for non-CCR5 tropic (i.e. CXCR4 tropic, dual/mixed tropic) and maraviroc or any other CCR5 antagonist would not be expected to be of therapeutic benefit to these HIV patients.

In a yet further embodiment of the invention, the viral population of the HIV patient contains more than 2% CXCR4 virus. In a yet further embodiment of the invention, the viral population of the HIV patient contains more than 5% CXCR4 virus. In a yet further embodiment of the invention, the viral population of the HIV patient contains more than 10% CXCR4 virus. In a yet further embodiment of the invention, the viral population of the HIV patient contains more than 15% CXCR4 virus. In a yet further embodiment of the invention, the viral population of the HIV patient contains more than 20% CXCR4 virus. In a yet further embodiment of the invention, the viral population of the HIV patient contains more than 25% CXCR4 virus. In a yet further embodiment of the invention, the viral population of the HIV patient contains more than 30% CXCR4 virus. In a yet further embodiment of the invention, the viral population of the HIV patient contains more than 35% CXCR4 virus. In a yet further embodiment of the invention, the viral population of the HIV patient contains more than 40% CXCR4 virus. In a yet further embodiment of the invention, the viral population of the HIV patient contains more than 45% CXCR4 virus. In a yet further embodiment of the invention, the viral population of the HIV patient contains more than 50% CXCR4 virus.

In a yet further embodiment, the CCR5 antagonist has an IC50 for CCR5 binding of less than 1 μM (as determined by the MIP-1βassay of Combadiere et al, J. Leukoc. Biol., 60, 147-152 (1996)).

In a yet further embodiment of the invention, the CCR5 antagonist is selected from maraviroc, preferably the free base of maraviroc, NCB-9471, PRO-140, CCR5 mAb004, TAK-779 (WO 99/32468), TAK-220 (WO 01/25200), TAK-652 which is disclosed in WO03014105 and has the chemical name 8-[4-(2-butoxyethoxy)phenyl]-1-isobutyl-N-[4-[[(1-propyl-1H-imadazol-5-yl)methyl]sulphinyl]phenyl]-1,2,3,4-tetrahydro-1-benzacocine-5-carboxamide, SC-351125, ancriviroc (formerly known as SCH-C), vicroviroc which has the chemical name (4,6-dimethylprymidine-5-yl){4-[(3S)-4-{(1R)-2-methoxy-1-[4-(trifluoromethyl)phenyl]ethyl}-3-methylpiperazin-1-yl]-4-methylpiperidin-1-yl}methanone, PRO-140, apliviroc (formerly known as GW-873140, Ono-4128, AK-602), AMD-887, INC-B9471, CMPD-167 which has the chemical name N-methyl-N-((1R,3S,4S)-3-[4-(3-benzyl-1-ethyl-1H-pyrazol-5-yl)piperidin-1-ylmethyl]-4-[3-fluorophenyl]cyclapent-1-yl]-D-valine), methyl1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carboxylate, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate and N-{(1S)-3-[3-endo-(5-isobutyryl-2-methyl-4,5,6,7-tetrahydro-1H-imidazo[4,5-c]pyridin-1-yl)-8-azabicyclo[3.2.1]oct-8-yl]-1-(3-fluorophenyl)propyl}acetamide) and pharmaceutically acceptable salts, solvates or derivatives of the above. The last four compounds are disclosed in WO 03/084954 and WO 05/033107, and methods to make them are disclosed therein.

In a yet further embodiment, the CCR5 antagonist is selected from maraviroc, vicriviroc, NCB-9471, PRO-140, CCR5 mAb004, 8-[4-(2-butoxyethoxy)phenyl]-1-isobutyl-N-[4-[[(1-propyl-1H-imadazol-5-yl)methyl]sulphinyl]phenyl]-1,2,3,4-tetrahydro-1-benzacocine-5-carboxamide, methyl1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carboxylate, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, and N-{(1S)-3-[3-endo-(5-isobutyryl-2-methyl-4,5,6,7-tetrahydro-1H-imidazo[4,5-c]pyridin-1-yl)-8-azabicyclo[3.2.1]oct-8-yl]-1-(3-fluorophenyl)propyl}acetamide) and pharmaceutically acceptable salts, solvates or derivatives of the above.

In a yet further embodiment, the CCR5 antagonist is maraviroc, preferably as the free base.

Additional CCR5 antagonists for use in the present invention can be identified by the ability of a selected compound, pharmaceutically acceptable salt, solvate or derivative thereof, to modulate chemokine receptor activity, which is demonstrated by methodology known in the art, such as by using the assay for CCR5 binding following procedures disclosed in Combadiere et al., J. Leukoc. Biol., 60, 147-52 (1996); and/or by using the intracellular calcium mobilisation assays as described by the same authors. Cell lines expressing the receptor of interest include those naturally expressing the receptor, such as PM-1, or IL-2 stimulated peripheral blood lymphocytes (PBL), or a cell engineered to express a recombinant receptor, such as CHO, 300.19, L1.2 or HEK-293.

Protease inhibitors (PI) and nucleoside/nucleotide reverse transcriptase inhibitors (NRTI) are known to have the side effect of adversely affecting a patient's lipid levels.

In another aspect of the invention, there is provided the use of a CCR5 antagonist in the preparation of a medicament to elevate HDL particles in an HIV patient who is taking a protease inhibitor or a nucleoside/nucleotide reverse transcriptase inhibitor.

Examples of PIs include, but are not limited to, amprenavir (141W94), CGP-73547, CGP-61755, DMP-450 (mozenavir), nelfinavir, ritonavir, saquinavir, lopinavir, TMC-126, atazanavir, palinavir, GS-3333, KN I-413, KNI-272, LG-71350, CGP-61755, PD 173606, PD 177298, PD 178390, PD 178392, U-140690, ABT-378, DMP-450, AG-1776, MK-944, VX-478, indinavir, tipranavir, TMC-114, DPC-681, DPC-684, fosamprenavir calcium, benzenesulfonamide derivatives disclosed in WO 03/053435, R-944, Ro-03-34649, VX-385, GS-224338, OPT-TL3, PL-100, PPL-100, SM-309515, AG-148, DG-35-VIII, DMP-850, GW-5950X, KNI-1039, L-756423, LB-71262, LP-130, RS-344, SE-063, UIC-94-003, Vb-19038, A-77003, BMS-182193, BMS-186318, SM-309515, JE-2147, GS-9005.

Examples of NRTIs include, but are not limited to, abacavir, GS-840, lamivudine, adefovir dipivoxil, beta-fluoro-ddA, zalcitabine, didanosine, stavudine, zidovudine, tenofovir disoproxil fumarate, amdoxovir (DAPD), SPD-754, SPD-756, racivir, reverset (DPC-817), MIV-210 (FLG), beta-L-Fd4C (ACH-126443), MIV-310 (alovudine, FLT), dOTC, DAPD, entecavir, GS-7340, emtricitabine (FTC).

In one embodiment of the invention, maraviroc is administered in combination with zidovudine and lamivudine.

The CCR5 antagonists may be used either alone or in combination with another pharmaceutical agent described herein, in the treatment of the following diseases/conditions, such as dyslipidemia, hypercholesterolemia, hypertriglyceridemia, peripheral vascular disease, cardiovascular disorders, angina, ischemia, cardiac ischemia, stroke, myocardial infarction, reperfusion injury, angioplastic restenosis, vascular complications of diabetes, unstable angina pectoris, Alzheimer's Disease, cerebrovacular disease, coronary artery disease and ventricular dysfunction.

A combination of the invention may be part of a pharmaceutical composition further containing a pharmaceutically active carrier, diluent, solvent or vehicle, each as described herein.

Examples of a suitable pharmaceutically active agent include a HMG-CoA reductase inhibitor, a cholesterol synthesis inhibitor, a cholesterol absorption inhibitor, another CETP inhibitor, a MTP/Apo B secretion inhibitor, a PPAR modulator and other cholesterol lowering agents such as a fibrate, niacin, an ion-exchange resin, an antioxidant, an ACAT inhibitor, and a bile acid sequestrant. Other pharmaceutical agents would also include the following: a bile acid reuptake inhibitor, an ileal bile acid transporter inhibitor, an ACC inhibitor, an antihypertensive (such as NORVASC®), a selective estrogen receptor modulator, a selective androgen receptor modulator, an antibiotic, an antidiabetic (such as metformin, a PPARγ activator, a sulfonylurea, insulin, an aldose reductase inhibitor (ARI) and a sorbitol dehydrogenase inhibitor (SDI)), an anti-obesity compound, a thyromimetic agent, an Alzheimer's disease drug and aspirin (acetylsalicylic acid or a nitric oxide releasing asprin). As used herein, “niacin” includes all available forms such as immediate release, slow release, extended release and low-flushing niacin. Niacin may also be combined with other therapeutic agents such as prostaglandins and/or statins, i.e. lovastatin or simvastatin, which are an HMG-CoA reductase inhibitor and described further below. This combination therapy is known as ADVICOR® (Kos Pharmaceuticals Inc.) In combination therapy treatment, both the compounds of this invention and the other drug therapies are administered to mammals (e.g., humans, male or female) by conventional methods.

In combination therapy treatment, the CCR5 antagonists and the other drug therapies are administered to mammals by conventional methods. The following discussion more specifically describes the various combination aspects of this invention.

The conversion of 3-hydroxy-3-methylglutaryl-coenzyme A (HMG-CoA) to mevalonate is an early and rate-limiting step in the cholesterol biosynthetic pathway. This step is catalyzed by the enzyme HMG-CoA reductase. Statins inhibit HMG-CoA reductase from catalyzing this conversion. Exemplary statins include lovastatin, simvastatin, pravastatin, fluvastatin, atorvastatin, rivastatin, rosuvastatin, pitavastatin, (3R,5R)-7-(4-(benzylcarbamoyl)-2-(4-fluorophenyl)-5-isopropyl-1H-imidazol-1-yl)-3,5-dihydroxyheptanoic acid; (3R,5R)-7-(4-((4-methylbenzyl)carbamoyl)-2-(4-fluorophenyl)-5-isopropyl-1H-pyrazol-1-yl)-3,5-dihydroxyheptanoic acid; and (3R,5R)-7-(4-((3-fluorobenzyl)carbamoyl)-5-cyclopropyl-2-(4-fluorophenyl)-1H-imidazol-1-yl)-3,5-dihydroxyheptanoic acid, and pharmaceutically acceptable salts thereof.

Atorvastatin calcium (i.e., atorvastatin hemicalcium), disclosed in U.S. Pat. No. 5,273,995, which is incorporated herein by reference, is currently sold as Lipitor® and has the formula

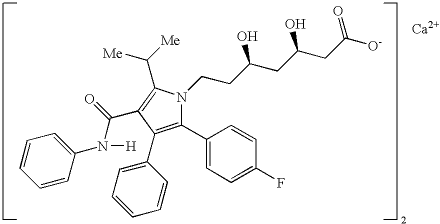

Atorvastatin calcium is a selective, competitive inhibitor of HMG-CoA. As such, atorvastatin calcium is a potent lipid lowering compound. The free carboxylic acid form of atorvastatin exists predominantly as the lactone of the formula

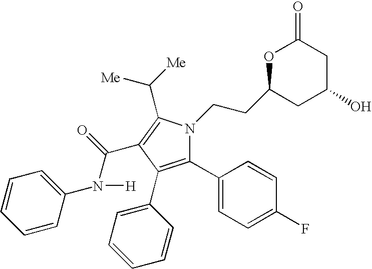

and is disclosed in U.S. Pat. No. 4,681,893, which is incorporated herein by reference.

Statins include such compounds as rosuvastatin disclosed in U.S. RE37,314 E, pitivastatin disclosed in EP 304063 B1 and U.S. Pat. No. 5,011,930, simvastatin, disclosed in U.S. Pat. No. 4,444,784, which is incorporated herein by reference; pravastatin, disclosed in U.S. Pat. No. 4,346,227 which is incorporated herein by reference; cerivastatin, disclosed in U.S. Pat. No. 5,502,199, which is incorporated herein by reference; mevastatin, disclosed in U.S. Pat. No. 3,983,140, which is incorporated herein by reference; velostatin, disclosed in U.S. Pat. No. 4,448,784 and U.S. Pat. No. 4,450,171, both of which are incorporated herein by reference; fluvastatin, disclosed in U.S. Pat. No. 4,739,073, which is incorporated herein by reference; compactin, disclosed in U.S. Pat. No. 4,804,770, which is incorporated herein by reference; lovastatin, disclosed in U.S. Pat. No. 4,231,938, which is incorporated herein by reference; dalvastatin, disclosed in European Patent Application Publication No. 738510 A2; fluindostatin, disclosed in European Patent Application Publication No. 363934 A1; atorvastatin, disclosed in U.S. Pat. No. 4,681,893, which is incorporated herein by reference; atorvastatin calcium (which is the hemicalcium salt of atorvastatin), disclosed in U.S. Pat. No. 5,273,995, which is incorporated herein by reference; and dihydrocompactin, disclosed in U.S. Pat. No. 4,450,171, which is incorporated herein by reference.

Further HMG CoA reductase inhibitors are disclosed in International Publication Nos. WO 2005/105079; and PCT/IB2005/003461 filed Nov. 14, 2005 (the disclosures of which are hereby incorporated by reference) including (3R,5R)-7-(4-(benzylcarbamoyl)-2-(4-fluorophenyl)-5-isopropyl-1H-imidazol-1-yl)-3,5-dihydroxyheptanoic acid; (3R,5R)-7-(4-((3-fluorobenzyl)carbamoyl)-5-cyclopropyl-2-(4-fluorophenyl)-1H-imidazol-1-yl)-3,5-dihydroxyheptanoic acid; and (3R,5R)-7-(4-((4-methylbenzyl)carbamoyl)-2-(4-fluorophenyl)-5-isopropyl-1H-pyrazol-1-yl)-3,5-dihydroxyheptanoic acid and pharmaceutically acceptable salts of said compounds.

Any PPAR modulator may be used in the combination aspect of this invention. The term PPAR modulator refers to compounds which modulate peroxisome proliferator activator receptor (PPAR) activity in mammals, particularly humans. Such modulation is readily determined by those skilled in the art according to standard assays known in the literature. It is believed that such compounds, by modulating the PPAR receptor, regulate transcription of key genes involved in lipid and glucose metabolism such as those in fatty acid oxidation and also those involved in high density lipoprotein (HDL) assembly (for example, apolipoprotein Al gene transcription), accordingly reducing whole body fat and increasing HDL cholesterol. By virtue of their activity, these compounds also reduce plasma levels of triglycerides, VLDL cholesterol, LDL cholesterol and their associated components such as apolipoprotein B in mammals, particularly humans, as well as increasing HDL cholesterol and apolipoprotein Al. Hence, these compounds are useful for the treatment and correction of the various dyslipidemias observed to be associated with the development and incidence of atherosclerosis and cardiovascular disease, including hypoalphalipoproteinemia and hypertriglyceridemia. A variety of these compounds are described and referenced below, however, others will be known to those skilled in the art. International Publication Nos. WO 2004/048334; WO 2005/092845; and WO 2006/003495 (the disclosures of which are hereby incorporated by reference) disclose certain compounds which are PPARα activators including 3-[3-(1-Carboxy-1-methyl-ethoxy)-phenyl]-piperidine-1-carboxylic acid 3-trifluoromethyl-benzyl ester; 3-[3-(1-Carboxy-1-methyl-ethoxy)-phenyl]-piperidine-1-carboxylic acid 4-trifluoromethyl-benzyl ester; 5-[4-(4-Ethyl-benzylsulfanyl)-phenylsulfamoyl]-2-methyl-benzoic acid; and 5-{2-[4-(3,4-Difluoro-phenoxy)-phenyl]-ethylsulfamoyl}-2-methyl-benzoic acid; and pharmaceutically acceptable salts of said compounds.

Any other PPAR modulator may be used in the combination aspect of this invention. In particular, modulators of PPARβ and/or PPARγ may be useful in combination with compounds of the present invention. Exemplary PPAR inhibitors are described in International Publication No. WO 2003/084916 as {5-Methoxy-2-methyl-4-[4-(4-trifluoromethyl-benzyloxy)-benzylsulfany]-phenoxy}-acetic acid and {5-Methoxy-2-methyl-4-[4-(5-trifluoromethyl-pyridin-2-yl)-benzylsulfanyl]-phenoxy}-acetic acid; and pharmaceutically acceptable salts of said compounds.

Any MTP/Apo B (microsomal triglyceride transfer protein and or apolipoprotein B) secretion inhibitor may be used in the combination aspect of this invention. The term MTP/Apo B secretion inhibitor refers to compounds which inhibit the secretion of triglycerides, cholesteryl ester, and phospholipids. Such inhibition is readily determined by those skilled in the art according to standard assays (e.g., Wetterau, J. R. 1992; Science 258:999). A variety of these compounds are described and referenced below however other MTP/Apo B secretion inhibitors will be known to those skilled in the art, including implitapide (Bayer) and additional compounds such as those disclosed in WO 96/40640 and WO 98/23593, (two exemplary publications).

For example, the following MTP/Apo B secretion inhibitors are particularly useful:
4′-trifluoromethyl-biphenyl-2-carboxylic acid [2-(1H-[1,2,4,]triazol-3-ylmethyl)-1,2,3,4-tetrahydro-isoquinolin-6-yl]-amide;
4′-trifluoromethyl-biphenyl-2-carboxylic acid [2-(2-acetylamino-ethyl)-1,2,3,4-tetrahydro-isoquinolin-6-yl]-amide;
(2-{6-[(4′-trifluoromethyl-biphenyl-2-carbonyl)-amino]-3,4-dihydro-1H-isoquinolin-2-yl}-ethyl)-carbamic acid methyl ester;
4′-trifluoromethyl-biphenyl-2-carboxylic acid [2-(1H-imidazol-2-ylmethyl)-1,2,3,4-tetrahydro-isoquinolin-6-yl]-amide;
4′-trifluoromethyl-biphenyl-2-carboxylic acid [2-(2,2-diphenylethyl)-1,2,3,4-tetrahydro-isoquinolin-6-yl]-amide;
4′-trifluoromethyl-biphenyl-2-carboxylic acid [2-(2-ethoxy-ethyl)-1,2,3,4-tetrahydro-isoquinolin-6-yl]-amide;
(S)—N-{2-[benzyl(methyl)amino]-2-oxo-1-phenylethyl}-1-methyl-5-[4′-(trifluoromethyl)[1,1′-biphenyl]-2-carboxamido]-1H-indole-2-carboxamide;
(S)-2-[(4′-Trifluoromethyl-biphenyl-2-carbonyl)-amino]-quinoline-6-carboxylic acid (pentylcarbamoyl-phenyl-methyl)-amide;
1H-indole-2-carboxamide,1-methyl-N-[(1S)-2-[methyl(phenylmethyl)amino]-2-oxo-1-phenylethyl]-5-[[[4′-(trifluoromethyl)[1,1′-biphenyl]-2-yl]carbonyl]amino]; and
N-[(1S)-2-(benzylmethylamino)-2-oxo-1-phenylethyl]-1-methyl-5-[[[4′-(trifluoromethyl)biphenyl-2-yl]carbonyl]amino]-1H-indole-2-carboxamide.

Any HMG-CoA synthase inhibitor may be used in the combination aspect of this invention. The term HMG-CoA synthase inhibitor refers to compounds which inhibit the biosynthesis of hydroxymethylglutaryl-coenzyme A from acetyl-coenzyme A and acetoacetyl-coenzyme A, catalyzed by the enzyme HMG-CoA synthase. Such inhibition is readily determined by those skilled in the art according to standard assays (Meth Enzymol. 1975; 35:155-160: Meth. Enzymol. 1985; 110:19-26 and references cited therein). A variety of these compounds are described and referenced below, however other HMG-CoA synthase inhibitors will be known to those skilled in the art. U.S. Pat. No. 5,120,729 (the disclosure of which is hereby incorporated by reference) discloses certain beta-lactam derivatives. U.S. Pat. No. 5,064,856 (the disclosure of which is hereby incorporated by reference) discloses certain spiro-lactone derivatives prepared by culturing a microorganism (MF5253). U.S. Pat. No. 4,847,271 (the disclosure of which is hereby incorporated by reference) discloses certain oxetane compounds such as 11-(3-hydroxymethyl-4-oxo-2-oxetayl)-3,5,7-trimethyl-2,4-undeca-dienoic acid derivatives.

Any compound that decreases HMG-CoA reductase gene expression may be used in the combination aspect of this invention. These agents may be HMG-CoA reductase transcription inhibitors that block the transcription of DNA or translation inhibitors that prevent or decrease translation of mRNA coding for HMG-CoA reductase into protein. Such compounds may either affect transcription or translation directly, or may be biotransformed to compounds that have the aforementioned activities by one or more enzymes in the cholesterol biosynthetic cascade or may lead to the accumulation of an isoprene metabolite that has the aforementioned activities. Such compounds may cause this effect by decreasing levels of SREBP (sterol receptor binding protein) by inhibiting the activity of site-1 protease (S1P) or agonizing the oxzgenal receptor or SCAP. Such regulation is readily determined by those skilled in the art according to standard assays (Meth. Enzymol. 1985; 110:9-19). Several compounds are described and referenced below, however other inhibitors of HMG-CoA reductase gene expression will be known to those skilled in the art. U.S. Pat. No. 5,041,432 (the disclosure of which is incorporated by reference) discloses certain 15-substituted lanosterol derivatives. Other oxygenated sterols that suppress synthesis of HMG-CoA reductase are discussed by E. I. Mercer (Prog. Lip. Res. 1993; 32:357-416).

Any compound having activity as a CETP inhibitor can serve in the combination therapy aspect of the present invention. The term CETP inhibitor refers to compounds that inhibit the cholesteryl ester transfer protein (CETP) mediated transport of various cholesteryl esters and triglycerides from HDL to LDL and VLDL. Such CETP inhibition activity is readily determined by those skilled in the art according to standard assays (e.g., U.S. Pat. No. 6,140,343). A variety of CETP inhibitors will be known to those skilled in the art, for example, those disclosed in commonly assigned U.S. Pat. No. 6,140,343 and commonly assigned U.S. Pat. Nos. 6,197,786 and 6,723,752. CETP inhibitors disclosed in these patents include compounds, such as (2R)-3-{[3-(4-Chloro-3-ethyl-phenoxy)-phenyl]-[[3-(1,1,2,2-tetrafluoro-ethoxy)-phenyl]-methyl]-amino}-1,1,1-trifluoro-2-propanol Moreover, CETP inhibitors included herein are also described in WO 2007/105050; WO 2007/105049; WO 2006/056854; WO 2006/014357; WO 2006/014413; WO2007/005572; WO2007/079186; WO 2007/047591; WO 2007/081571; U.S. Pat. No. 6,426,365; and WO 2004/20393. U.S. Pat. No. 5,512,548 discloses certain polypeptide derivatives having activity as CETP inhibitors, while certain CETP-inhibitory rosenonolactone derivatives and phosphate-containing analogs of cholesteryl ester are disclosed in J. Antibiot., 49(8): 815-816 (1996), and Bioorg. Med. Chem. Lett.; 6:1951-1954 (1996), respectively.

Exemplary CETP inhibitors include 2-methyl-, S-[2[[[1-(2-ethylbutyl)cyclohexyl]carbonyl]amino]phenyl]ester propanethioic acid as described in U.S. Pat. No. 6,426,365; trans-(4-{[N-(2-{[N′-[3,5-bis(trifluoromethyl)benzyl]-N′-(2-methyl-2H-tetrazol-5-yl)amino]methyl}-5-methyl-4-trifluoromethylphenyl)-N-ethylamino]methyl}cyclohexyl)acetic acid methanesulfonate as described in WO 2004/20393; (4S,5R)-5-[3,5-bis(trifluoromethyl)phenyl]-3-{[4′-fluoro-5′ isopropyl-2′-methoxy-4-(trifluoromethyl)biphenyl-2-yl]methyl}-4-methyl-1,3-oxazolidin-2-one as described in WO 2006/014357, WO 2006/014413, and WO 2007/005572; S-[2-[1-(2-ethylbutyl)cyclohexanecarbonylamino]-phenyl]2-methylthiopropionatecis-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide as described in WO 2006/033002; and (2R)-3-{[3-(4-Chloro-3-ethyl-phenoxy)-phenyl][[3-(1,1,2,2-tetrafluoro-ethoxy)-phenyl]-methyl]-amino}-1,1,1-trifluoro-2-propanol as described in U.S. Pat. No. 6,723,752 or a pharmaceutically acceptable salt of said compounds.

Any squalene synthetase inhibitor may be used in the combination aspect of this invention. The term squalene synthetase inhibitor refers to compounds which inhibit the condensation of 2 molecules of farnesylpyrophosphate to form squalene, catalyzed by the enzyme squalene synthetase. Such inhibition is readily determined by those skilled in the art according to standard assays (Meth. Enzymol. 1969; 15: 393-454 and Meth. Enzymol. 1985; 110:359-373 and references contained therein). A variety of these compounds are described in and referenced below however other squalene synthetase inhibitors will be known to those skilled in the art. U.S. Pat. No. 5,026,554 (the disclosure of which is incorporated by reference) discloses fermentation products of the microorganism MF5465 (ATCC 74011) including zaragozic acid. A summary of other patented squalene synthetase inhibitors has been compiled (Curr. Op. Ther. Patents (1993) 861-4).

Any squalene epoxidase inhibitor may be used in the combination aspect of this invention. The term squalene epoxidase inhibitor refers to compounds which inhibit the bioconversion of squalene and molecular oxygen into squalene-2,3-epoxide, catalyzed by the enzyme squalene epoxidase. Such inhibition is readily determined by those skilled in the art according to standard assays (Biochim. Biophys. Acta 1984; 794:466-471). A variety of these compounds are described and referenced below, however other squalene epoxidase inhibitors will be known to those skilled in the art. U.S. Pat. Nos. 5,011,859 and 5,064,864 (the disclosures of which are incorporated by reference) disclose certain fluoro analogs of squalene. EP publication 395,768 A (the disclosure of which is incorporated by reference) discloses certain substituted allylamine derivatives. PCT publication WO 9312069 A (the disclosure of which is hereby incorporated by reference) discloses certain amino alcohol derivatives, U.S. Pat. Nos. 6,110,909, 6,613,761, which include 2-(1-(2-((3S,6S)-1-(3-acetoxy-2,2-dimethylpropyl)-8-chloro-6-(2,3-dimethoxyphenyl)-2-oxo-2,3,4,6-tetrahydro-1H-benzo[c][1,5]oxazocin-3-yl)acetyl)piperidin-4-yl)acetic acid. U.S. Pat. No. 5,051,534 (the disclosure of which is hereby incorporated by reference) discloses certain cyclopropyloxy-squalene derivatives.

Any squalene cyclase inhibitor may be used as the second component in the combination aspect of this invention. The term squalene cyclase inhibitor refers to compounds which inhibit the bioconversion of squalene-2,3-epoxide to lanosterol, catalyzed by the enzyme squalene cyclase. Such inhibition is readily determined by those skilled in the art according to standard assays (FEBS Lett. 1989; 244:347-350.). In addition, the compounds described and referenced below are squalene cyclase inhibitors, however other squalene cyclase inhibitors will also be known to those skilled in the art. PCT publication WO9410150 (the disclosure of which is hereby incorporated by reference) discloses certain 1,2,3,5,6,7,8,8a-octahydro-5,5,8(beta)-trimethyl-6-isoquinolineamine derivatives, such as N-trifluoroacetyl-1,2,3,5,6,7,8,8a-octahydro-2-allyl-5,5,8(beta)-trimethyl-6(beta)-isoquinolineamine. French patent publication 2697250 (the disclosure of which is hereby incorporated by reference) discloses certain beta,beta-dimethyl-4-piperidine ethanol derivatives such as 1-(1,5,9-trimethyldecyl)-beta,beta-dimethyl-4-piperidineethanol.

Any combined squalene epoxidase/squalene cyclase inhibitor may be used as the second component in the combination aspect of this invention. The term combined squalene epoxidase/squalene cyclase inhibitor refers to compounds that inhibit the bioconversion of squalene to lanosterol via a squalene-2,3-epoxide intermediate. In some assays it is not possible to distinguish between squalene epoxidase inhibitors and squalene cyclase inhibitors, however, these assays are recognized by those skilled in the art. Thus, inhibition by combined squalene epoxidase/squalene cyclase inhibitors is readily determined by those skilled in art according to the aforementioned standard assays for squalene cyclase or squalene epoxidase inhibitors. A variety of these compounds are described and referenced below, however other squalene epoxidase/squalene cyclase inhibitors will be known to those skilled in the art. U.S. Pat. Nos. 5,084,461 and 5,278,171 (the disclosures of which are incorporated by reference) disclose certain azadecalin derivatives. EP publication 468,434 (the disclosure of which is incorporated by reference) discloses certain piperidyl ether and thio-ether derivatives such as 2-(1-piperidyl)pentyl isopentyl sulfoxide and 2-(1-piperidyl)ethyl ethyl sulfide. PCT publication WO 9401404 (the disclosure of which is hereby incorporated by reference) discloses certain acyl-piperidines such as 1-(1-oxopentyl-5-phenylthio)-4-(2-hydroxy-1-methyl)-ethyl)piperidine. U.S. Pat. No. 5,102,915 (the disclosure of which is hereby incorporated by reference) discloses certain cyclopropyloxy-squalene derivatives.

The compounds of the present invention may also be administered in combination with naturally occurring compounds that act to lower plasma cholesterol levels. These naturally occurring compounds are commonly called nutraceuticals and include, for example, garlic extract and niacin. A slow-release form of niacin is available and is known as Niaspan. Niacin may also be combined with other therapeutic agents such as lovastatin, or another is an HMG-CoA reductase inhibitor. This combination therapy with lovastatin is known as ADVICOR™ (Kos Pharmaceuticals Inc.).

Any cholesterol absorption inhibitor can be used as an additional in the combination aspect of the present invention. The term cholesterol absorption inhibition refers to the ability of a compound to prevent cholesterol contained within the lumen of the intestine from entering into the intestinal cells and/or passing from within the intestinal cells into the lymph system and/or into the blood stream. Such cholesterol absorption inhibition activity is readily determined by those skilled in the art according to standard assays (e.g., J. Lipid Res. (1993) 34: 377-395). Cholesterol absorption inhibitors are known to those skilled in the art and are described, for example, in PCT WO 94/00480. An example of a recently approved cholesterol absorption inhibitor is ZETIA™ (ezetimibe) (Schering-Plough/Merck). Other cholesterol absorption inhibitors currently in development include those disclosed in U.S. Pat. Nos. 7,205,290 and 6,992,067.

Any ACAT inhibitor may be used in the combination therapy aspect of the present invention. The term ACAT inhibitor refers to compounds that inhibit the intracellular esterification of dietary cholesterol by the enzyme acyl CoA: cholesterol acyltransferase. Such inhibition may be determined readily by one of skill in the art according to standard assays, such as the method of Heider et al. described in Journal of Lipid Research., 24:1127 (1983). A variety of these compounds are known to those skilled in the art, for example, U.S. Pat. No. 5,510,379 discloses certain carboxysulfonates, while WO 96/26948 and WO 96/10559 both disclose urea derivatives having ACAT inhibitory activity. Examples of ACAT inhibitors include compounds such as Avasimibe (Pfizer), CS-505 (Sankyo) and Eflucimibe (Eli Lilly and Pierre Fabre).

A lipase inhibitor may be used in the combination therapy aspect of the present invention. A lipase inhibitor is a compound that inhibits the metabolic cleavage of dietary triglycerides or plasma phospholipids into free fatty acids and the corresponding glycerides (e.g. EL, HL, etc.). Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a glyceride and fatty acid. In the intestine, the resultant free fatty acids and monoglycerides are incorporated into bile acid-phospholipid micelles, which are subsequently absorbed at the level of the brush border of the small intestine. The micelles eventually enter the peripheral circulation as chylomicrons. Such lipase inhibition activity is readily determined by those skilled in the art according to standard assays (e.g., Methods Enzymol. 286: 190-231).

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors have utility in the treatment of obesity and the other related conditions. Such pancreatic lipase inhibition activity is readily determined by those skilled in the art according to standard assays (e.g., Methods Enzymol, 286: 190-231).

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. See, for example, C. K. Abrams, et al., Gastroenterology, 92,125 (1987). Such gastric lipase inhibition activity is readily determined by those skilled in the art according to standard assays (e.g., Methods Enzymol. 286: 190-231).

A variety of gastric and/or pancreatic lipase inhibitors are known to one of ordinary skill in the art. Preferred lipase inhibitors are those inhibitors that are selected from the group consisting of lipstatin, tetrahydrolipstatin (orlistat), valilactone, esterastin, ebelactone A, and ebelactone B. The compound tetrahydrolipstatin is especially preferred. The lipase inhibitor, N-3-trifluoromethylphenyl-N′-3-chloro-4′-trifluoromethylphenylurea, and the various urea derivatives related thereto, are disclosed in U.S. Pat. No. 4,405,644. The lipase inhibitor, esteracin, is disclosed in U.S. Pat. Nos. 4,189,438 and 4,242,453. The lipase inhibitor, cyclo-O,O′-[(1,6-hexanediyl)-bis-(iminocarbonyl)]dioxime, and the various bis(iminocarbonyl)dioximes related thereto may be prepared as described in Petersen et al., Liebig's Annalen, 562, 205-229 (1949).

A variety of pancreatic lipase inhibitors are described herein below. The pancreatic lipase inhibitors lipstatin, (2S,3S,5S,7Z,10Z)-5-[(S)-2-formamido-4-methyl-valeryloxy]-2-hexyl-3-hydroxy-7,10-hexadecanoic acid lactone, and tetrahydrolipstatin (orlistat), (2S,3S,5S)-5-[(S)-2-formamido-4-methyl-valeryloxy]-2-hexyl-3-hydroxy-hexadecanoic 1,3 acid lactone, and the variously substituted N-formylleucine derivatives and stereoisomers thereof, are disclosed in U.S. Pat. No. 4,598,089. For example, tetrahydrolipstatin is prepared as described in, e.g., U.S. Pat. Nos. 5,274,143; 5,420,305; 5,540,917; and 5,643,874. The pancreatic lipase inhibitor, FL-386, 1-[4-(2-methylpropyl)cyclohexyl]-2-[(phenylsulfonyl)oxy]-ethanone, and the variously substituted sulfonate derivatives related thereto, are disclosed in U.S. Pat. No. 4,452,813. The pancreatic lipase inhibitor, WAY-121898, 4-phenoxyphenyl-4-methylpiperidin-1-yl-carboxylate, and the various carbamate esters and pharmaceutically acceptable salts related thereto, are disclosed in U.S. Pat. Nos. 5,512,565; 5,391,571 and 5,602,151. The pancreatic lipase inhibitor, valilactone, and a process for the preparation thereof by the microbial cultivation of Actinomycetes strain MG147-CF2, are disclosed in Kitahara, et al., J. Antibiotics, 40 (11), 1647-1650 (1987). The pancreatic lipase inhibitors, ebelactone A and ebelactone B, and a process for the preparation thereof by the microbial cultivation of Actinomycetes strain MG7-G1, are disclosed in Umezawa, et al., J. Antibiotics, 33, 1594-1596 (1980). The use of ebelactones A and B in the suppression of monoglyceride formation is disclosed in Japanese Kokai 08-143457, published Jun. 4, 1996.

Other compounds that are marketed for hyperlipidemia, including hypercholesterolemia and which are intended to help prevent or treat atherosclerosis include bile acid sequestrants, such as Welchol®, Colestid®, LoCholest® and Questran®; and fibric acid derivatives, such as Atromid®, Lopid® and Tricor®.

The compounds of the present invention can also be used in combination with other antihypertensive agents. Any anti-hypertensive agent can be used as the second agent in such combinations and examples are provided herein. Such antihypertensive activity is readily determined by those skilled in the art according to standard assays (e.g., blood pressure measurements).

Examples of presently marketed products containing antihypertensive agents include calcium channel blockers, such as Cardizem®, Adalat®, Calan®, Cardene®, Covera®, Dilacor®, DynaCirc® Procardia XL®, Sular®, Tiazac®, Vascor®, Verelan®, Isoptin®, Nimotop®. Norvasc®, and Plendil®; angiotensin converting enzyme (ACE) inhibitors, such as Accupril®, Altace®, Captopril®, Lotensin®, Mavik®, Monopril®, Prinivil®, Univasc®, Vasotec® and Zestril®.

Amlodipine and related dihydropyridine compounds are disclosed in U.S. Pat. Nos. 4,572,909 and 5,155,120, which are incorporated herein by reference, as potent anti-ischemic and antihypertensive agents. U.S. Pat. No. 4,879,303, which is incorporated herein by reference, discloses amlodipine benzenesulfonate salt (also termed amlodipine besylate). Amlodipine and amlodipine besylate are potent and long lasting calcium channel blockers. As such, amlodipine, amlodipine besylate, amlodipine maleate and other pharmaceutically acceptable acid addition salts of amlodipine have utility as antihypertensive agents and as antiischemic agents. Amlodipine besylate is currently sold as Norvasc®. Amlodipine has the formula

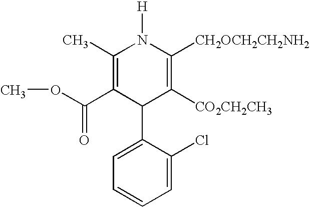

Calcium channel blockers which are within the scope of this invention include, but are not limited to: bepridil, which may be prepared as disclosed in U.S. Pat. No. 3,962,238 or U.S. Reissue No. 30,577; clentiazem, which may be prepared as disclosed in U.S. Pat. No. 4,567,175; diltiazem, which may be prepared as disclosed in U.S. Pat. No. 3,562, fendiline, which may be prepared as disclosed in U.S. Pat. No. 3,262,977; gallopamil, which may be prepared as disclosed in U.S. Pat. No. 3,261,859; mibefradil, which may be prepared as disclosed in U.S. Pat. No. 4,808,605; prenylamine, which may be prepared as disclosed in U.S. Pat. No. 3,152,173; semotiadil, which may be prepared as disclosed in U.S. Pat. No. 4,786,635; terodiline, which may be prepared as disclosed in U.S. Pat. No. 3,371,014; verapamil, which may be prepared as disclosed in U.S. Pat. No. 3,261,859; aranipine, which may be prepared as disclosed in U.S. Pat. No. 4,572,909; barnidipine, which may be prepared as disclosed in U.S. Pat. No. 4,220,649; benidipine, which may be prepared as disclosed in European Patent Application Publication No. 106,275; cilnidipine, which may be prepared as disclosed in U.S. Pat. No. 4,672,068; efonidipine, which may be prepared as disclosed in U.S. Pat. No. 4,885,284; elgodipine, which may be prepared as disclosed in U.S. Pat. No. 4,952,592; felodipine, which may be prepared as disclosed in U.S. Pat. No. 4,264,611; isradipine, which may be prepared as disclosed in U.S. Pat. No. 4,466,972; lacidipine, which may be prepared as disclosed in U.S. Pat. No. 4,801,599; lercanidipine, which may be prepared as disclosed in U.S. Pat. No. 4,705,797; manidipine, which may be prepared as disclosed in U.S. Pat. No. 4,892,875; nicardipine, which may be prepared as disclosed in U.S. Pat. No. 3,985,758; nifedipine, which may be prepared as disclosed in U.S. Pat. No. 3,485,847; nilvadipine, which may be prepared as disclosed in U.S. Pat. No. 4,338,322; nimodipine, which may be prepared as disclosed in U.S. Pat. No. 3,799,934; nisoldipine, which may be prepared as disclosed in U.S. Pat. No. 4,154,839; nitrendipine, which may be prepared as disclosed in U.S. Pat. No. 3,799,934; cinnarizine, which may be prepared as disclosed in U.S. Pat. No. 2,882,271; flunarizine, which may be prepared as disclosed in U.S. Pat. No. 3,773,939; lidoflazine, which may be prepared as disclosed in U.S. Pat. No. 3,267,104; lomerizine, which may be prepared as disclosed in U.S. Pat. No. 4,663,325; bencyclane, which may be prepared as disclosed in Hungarian Patent No. 151,865; etafenone, which may be prepared as disclosed in German Patent No. 1,265,758; and perhexyline, which may be prepared as disclosed in British Patent No. 1,025,578. The disclosures of all such U.S. Patents are incorporated herein by reference.

Angiotensin Converting Enzyme Inhibitors (ACE-Inhibitors) which are within the scope of this invention include, but are not limited to: alacepril, which may be prepared as disclosed in U.S. Pat. No. 4,248,883; benazepril, which may be prepared as disclosed in U.S. Pat. No. 4,410,520; captopril, which may be prepared as disclosed in U.S. Pat. Nos. 4,046,889 and 4,105,776; ceronapril, which may be prepared as disclosed in U.S. Pat. No. 4,452,790; delapril, which may be prepared as disclosed in U.S. Pat. No. 4,385,051; enalapril, which may be prepared as disclosed in U.S. Pat. No. 4,374,829; fosinopril, which may be prepared as disclosed in U.S. Pat. No. 4,337,201; imadapril, which may be prepared as disclosed in U.S. Pat. No. 4,508,727; lisinopril, which may be prepared as disclosed in U.S. Pat. No. 4,555,502; moveltopril, which may be prepared as disclosed in Belgian Patent No. 893,553; perindopril, which may be prepared as disclosed in U.S. Pat. No. 4,508,729; quinapril, which may be prepared as disclosed in U.S. Pat. No. 4,344,949; ramipril, which may be prepared as disclosed in U.S. Pat. No. 4,587,258; spirapril, which may be prepared as disclosed in U.S. Pat. No. 4,470,972; temocapril, which may be prepared as disclosed in U.S. Pat. No. 4,699,905; and trandolapril, which may be prepared as disclosed in U.S. Pat. No. 4,933,361. The disclosures of all such U.S. patents are incorporated herein by reference.

Angiotensin-II receptor antagonists (A-II antagonists) which are within the scope of this invention include, but are not limited to: candesartan, which may be prepared as disclosed in U.S. Pat. No. 5,196,444; eprosartan, which may be prepared as disclosed in U.S. Pat. No. 5,185,351; irbesartan, which may be prepared as disclosed in U.S. Pat. No. 5,270,317; losartan, which may be prepared as disclosed in U.S. Pat. No. 5,138,069; olmesartan and/or olmesartan medoxomil, which may be prepared as disclosed in U.S. Pat. No. 5,616,599; and valsartan, which may be prepared as disclosed in U.S. Pat. No. 5,399,578. The disclosures of all such U.S. patents are incorporated herein by reference.

Phosphodiesterase type 5 inhibitors (PDE5 inhibitors) which are within the scope of this invention include, but are not limited to: sildenafil, which may be prepared as disclosed in U.S. Pat. No. 5,250,534; and the PDE5 inhibitors disclosed in International Publication Numbers: WO2004096810, WO2005049616, WO2005049617, WO2006120552, WO 2007054778, and EP1348707.

Beta-adrenergic receptor blockers (beta- or β-blockers) which are within the scope of this invention include, but are not limited to: acebutolol, which may be prepared as disclosed in U.S. Pat. No. 3,857,952; alprenolol, which may be prepared as disclosed in Netherlands Patent Application No. 6,605,692; amosulalol, which may be prepared as disclosed in U.S. Pat. No. 4,217,305; arotinolol, which may be prepared as disclosed in U.S. Pat. No. 3,932,400; atenolol, which may be prepared as disclosed in U.S. Pat. No. 3,663,607 or 3,836,671; befunolol, which may be prepared as disclosed in U.S. Pat. No. 3,853,923; betaxolol, which may be prepared as disclosed in U.S. Pat. No. 4,252,984; bevantolol, which may be prepared as disclosed in U.S. Pat. No. 3,857,981; bisoprolol, which may be prepared as disclosed in U.S. Pat. No. 4,171,370; bopindolol, which may be prepared as disclosed in U.S. Pat. No. 4,340,541; bucumolol, which may be prepared as disclosed in U.S. Pat. No. 3,663,570; bufetolol, which may be prepared as disclosed in U.S. Pat. No. 3,723,476; bufuralol, which may be prepared as disclosed in U.S. Pat. No. 3,929,836; bunitrolol, which may be prepared as disclosed in U.S. Pat. Nos. 3,940,489 and 3,961,071; buprandolol, which may be prepared as disclosed in U.S. Pat. No. 3,309,406; butiridine hydrochloride, which may be prepared as disclosed in French Patent No. 1,390,056; butofilolol, which may be prepared as disclosed in U.S. Pat. No. 4,252,825; carazolol, which may be prepared as disclosed in German Patent No. 2,240,599; carteolol, which may be prepared as disclosed in U.S. Pat. No. 3,910,924; carvedilol, which may be prepared as disclosed in U.S. Pat. No. 4,503,067; celiprolol, which may be prepared as disclosed in U.S. Pat. No. 4,034,009; cetamolol, which may be prepared as disclosed in U.S. Pat. No. 4,059,622; cloranolol, which may be prepared as disclosed in German Patent No. 2,213,044; dilevalol, which may be prepared as disclosed in Clifton et al., Journal of Medicinal Chemistry, 1982, 25, 670; epanolol, which may be prepared as disclosed in European Patent Publication Application No. 41,491; indenolol, which may be prepared as disclosed in U.S. Pat. No. 4,045,482; labetalol, which may be prepared as disclosed in U.S. Pat. No. 4,012,444; levobunolol, which may be prepared as disclosed in U.S. Pat. No. 4,463,176; mepindolol, which may be prepared as disclosed in Seeman et al., Helv. Chim. Acta, 1971, 54, 241; metipranolol, which may be prepared as disclosed in Czechoslovakian Patent Application No. 128,471; metoprolol, which may be prepared as disclosed in U.S. Pat. No. 3,873,600; moprolol, which may be prepared as disclosed in U.S. Pat. No. 3,501,7691; nadolol, which may be prepared as disclosed in U.S. Pat. No. 3,935,267; nadoxolol, which may be prepared as disclosed in U.S. Pat. No. 3,819,702; nebivalol, which may be prepared as disclosed in U.S. Pat. No. 4,654,362; nipradilol, which may be prepared as disclosed in U.S. Pat. No. 4,394,382; oxprenolol, which may be prepared as disclosed in British Patent No. 1,077,603; perbutolol, which may be prepared as disclosed in U.S. Pat. No. 3,551,493; pindolol, which may be prepared as disclosed in Swiss Patent Nos. 469,002 and 472,404; practolol, which may be prepared as disclosed in U.S. Pat. No. 3,408,387; pronethalol, which may be prepared as disclosed in British Patent No. 909,357; propranolol, which may be prepared as disclosed in U.S. Pat. Nos. 3,337,628 and 3,520,919; sotalol, which may be prepared as disclosed in Uloth et al., Journal of Medicinal Chemistry, 1966, 9, 88; sufinalol, which may be prepared as disclosed in German Patent No. 2,728,641; talindol, which may be prepared as disclosed in U.S. Pat. Nos. 3,935,259 and 4,038,313; tertatolol, which may be prepared as disclosed in U.S. Pat. No. 3,960,891; tilisolol, which may be prepared as disclosed in U.S. Pat. No. 4,129,565; timolol, which may be prepared as disclosed in U.S. Pat. No. 3,655,663; toliprolol, which may be prepared as disclosed in U.S. Pat. No. 3,432,545; and xibenolol, which may be prepared as disclosed in U.S. Pat. No. 4,018,824. The disclosures of all such U.S. patents are incorporated herein by reference.

Alpha-adrenergic receptor blockers (alpha- or α-blockers) which are within the scope of this invention include, but are not limited to: amosulalol, which may be prepared as disclosed in U.S. Pat. No. 4,217,307; arotinolol, which may be prepared as disclosed in U.S. Pat. No. 3,932,400; dapiprazole, which may be prepared as disclosed in U.S. Pat. No. 4,252,721; doxazosin, which may be prepared as disclosed in U.S. Pat. No. 4,188,390; fenspiride, which may be prepared as disclosed in U.S. Pat. No. 3,399,192; indoramin, which may be prepared as disclosed in U.S. Pat. No. 3,527,761; labetolol; naftopidil, which may be prepared as disclosed in U.S. Pat. No. 3,997,666; nicergoline, which may be prepared as disclosed in U.S. Pat. No. 3,228,943; prazosin, which may be prepared as disclosed in U.S. Pat. No. 3,511,836; tamsulosin, which may be prepared as disclosed in U.S. Pat. No. 4,703,063; tolazoline, which may be prepared as disclosed in U.S. Pat. No. 2,161,938; trimazosin, which may be prepared as disclosed in U.S. Pat. No. 3,669,968; and yohimbine, which may be isolated from natural sources according to methods well known to those skilled in the art. The disclosures of all such U.S. patents are incorporated herein by reference.

The term “vasodilator,” where used herein, is meant to include cerebral vasodilators, coronary vasodilators and peripheral vasodilators. Cerebral vasodilators within the scope of this invention include, but are not limited to: bencyclane; cinnarizine; citicoline, which may be isolated from natural sources as disclosed in Kennedy et al., Journal of the American Chemical Society, 1955, 77, 250 or synthesized as disclosed in Kennedy, Journal of Biological Chemistry, 1956, 222, 185; cyclandelate, which may be prepared as disclosed in U.S. Pat. No. 3,663,597; ciclonicate, which may be prepared as disclosed in German Patent No. 1,910,481; diisopropylamine dichloroacetate, which may be prepared as disclosed in British Patent No. 862,248; eburnamonine, which may be prepared as disclosed in Hermann et al., Journal of the American Chemical Society, 1979, 101, 1540; fasudil, which may be prepared as disclosed in U.S. Pat. No. 4,678,783; fenoxedil, which may be prepared as disclosed in U.S. Pat. No. 3,818,021; flunarizine, which may be prepared as disclosed in U.S. Pat. No. 3,773,939; ibudilast, which may be prepared as disclosed in U.S. Pat. No. 3,850,941; ifenprodil, which may be prepared as disclosed in U.S. Pat. No. 3,509,164; lomerizine, which may be prepared as disclosed in U.S. Pat. No. 4,663,325; nafronyl, which may be prepared as disclosed in U.S. Pat. No. 3,334,096; nicametate, which may be prepared as disclosed in Blicke et al., Journal of the American Chemical Society, 1942, 64, 1722; nicergoline, which may be prepared as disclosed above; nimodipine, which may be prepared as disclosed in U.S. Pat. No. 3,799,934; papaverine, which may be prepared as reviewed in Goldberg, Chem. Prod. Chem. News, 1954, 17, 371; pentifylline, which may be prepared as disclosed in German Patent No. 860,217; tinofedrine, which may be prepared as disclosed in U.S. Pat. No. 3,563,997; vincamine, which may be prepared as disclosed in U.S. Pat. No. 3,770,724; vinpocetine, which may be prepared as disclosed in U.S. Pat. No. 4,035,750; and viquidil, which may be prepared as disclosed in U.S. Pat. No. 2,500,444. The disclosures of all such U.S. patents are incorporated herein by reference.

Coronary vasodilators within the scope of this invention include, but are not limited to: amotriphene, which may be prepared as disclosed in U.S. Pat. No. 3,010,965; bendazol, which may be prepared as disclosed in J. Chem. Soc. 1958, 2426; benfurodil hemisuccinate, which may be prepared as disclosed in U.S. Pat. No. 3,355,463; benziodarone, which may be prepared as disclosed in U.S. Pat. No. 3,012,042; chloracizine, which may be prepared as disclosed in British Patent No. 740,932; chromonar, which may be prepared as disclosed in U.S. Pat. No. 3,282,938; clobenfural, which may be prepared as disclosed in British Patent No. 1,160,925; clonitrate, which may be prepared from propanediol according to methods well known to those skilled in the art, e.g., see Annalen, 1870, 155, 165; cloricromen, which may be prepared as disclosed in U.S. Pat. No. 4,452,811; dilazep, which may be prepared as disclosed in U.S. Pat. No. 3,532,685; dipyridamole, which may be prepared as disclosed in British Patent No. 807,826; droprenilamine, which may be prepared as disclosed in German Patent No. 2,521,113; efloxate, which may be prepared as disclosed in British Patent Nos. 803,372 and 824,547; erythrityl tetranitrate, which may be prepared by nitration of erythritol according to methods well-known to those skilled in the art; etafenone, which may be prepared as disclosed in German Patent No. 1,265,758; fendiline, which may be prepared as disclosed in U.S. Pat. No. 3,262,977; floredil, which may be prepared as disclosed in German Patent No. 2,020,464; ganglefene, which may be prepared as disclosed in U.S.S.R. Patent No. 115,905; hexestrol, which may be prepared as disclosed in U.S. Pat. No. 2,357,985; hexobendine, which may be prepared as disclosed in U.S. Pat. No. 3,267,103; itramin tosylate, which may be prepared as disclosed in Swedish Patent No. 168,308; khellin, which may be prepared as disclosed in Baxter et al., Journal of the Chemical Society, 1949, S 30; lidoflazine, which may be prepared as disclosed in U.S. Pat. No. 3,267,104; mannitol hexanitrate, which may be prepared by the nitration of mannitol according to methods well-known to those skilled in the art; medibazine, which may be prepared as disclosed in U.S. Pat. No. 3,119,826; nitroglycerin; pentaerythritol tetranitrate, which may be prepared by the nitration of pentaerythritol according to methods well-known to those skilled in the art; pentrinitrol, which may be prepared as disclosed in German Patent No. 638, 422-3; perhexylline, which may be prepared as disclosed above; pimethylline, which may be prepared as disclosed in U.S. Pat. No. 3,350,400; prenylamine, which may be prepared as disclosed in U.S. Pat. No. 3,152,173; propatyl nitrate, which may be prepared as disclosed in French Patent No. 1,103,113; trapidil, which may be prepared as disclosed in East German Patent No. 55,956; tricromyl, which may be prepared as disclosed in U.S. Pat. No. 2,769,015; trimetazidine, which may be prepared as disclosed in U.S. Pat. No. 3,262,852; trolnitrate phosphate, which may be prepared by nitration of triethanolamine followed by precipitation with phosphoric acid according to methods well-known to those skilled in the art; visnadine, which may be prepared as disclosed in U.S. Pat. Nos. 2,816,118 and 2,980,699. The disclosures of all such U.S. patents are incorporated herein by reference.

Peripheral vasodilators within the scope of this invention include, but are not limited to: aluminum nicotinate, which may be prepared as disclosed in U.S. Pat. No. 2,970,082; bamethan, which may be prepared as disclosed in Corrigan et al., Journal of the American Chemical Society, 1945, 67, 1894; bencyclane, which may be prepared as disclosed above; betahistine, which may be prepared as disclosed in Walter et al.; Journal of the American Chemical Society, 1941, 63, 2771; bradykinin, which may be prepared as disclosed in Hamburg et al., Arch. Biochem. Biophys., 1958, 76, 252; brovincamine, which may be prepared as disclosed in U.S. Pat. No. 4,146,643; bufeniode, which may be prepared as disclosed in U.S. Pat. No. 3,542,870; buflomedil, which may be prepared as disclosed in U.S. Pat. No. 3,895,030; butalamine, which may be prepared as disclosed in U.S. Pat. No. 3,338,899; cetiedil, which may be prepared as disclosed in French Patent Nos. 1,460,571; ciclonicate, which may be prepared as disclosed in German Patent No. 1,910,481; cinepazide, which may be prepared as disclosed in Belgian Patent No. 730,345; cinnarizine, which may be prepared as disclosed above; cyclandelate, which may be prepared as disclosed above; diisopropylamine dichloroacetate, which may be prepared as disclosed above; eledoisin, which may be prepared as disclosed in British Patent No. 984,810; fenoxedil, which may be prepared as disclosed above; flunarizine, which may be prepared as disclosed above; hepronicate, which may be prepared as disclosed in U.S. Pat. No. 3,384,642; ifenprodil, which may be prepared as disclosed above; iloprost, which may be prepared as disclosed in U.S. Pat. No. 4,692,464; inositol niacinate, which may be prepared as disclosed in Badgett et al., Journal of the American Chemical Society, 1947, 69, 2907; isoxsuprine, which may be prepared as disclosed in U.S. Pat. No. 3,056,836; kallidin, which may be prepared as disclosed in Biochem. Biophys. Res. Commun., 1961, 6, 210; kallikrein, which may be prepared as disclosed in German Patent No. 1,102,973; moxisylyte, which may be prepared as disclosed in German Patent No. 905,738; nafronyl, which may be prepared as disclosed above; nicametate, which may be prepared as disclosed above; nicergoline, which may be prepared as disclosed above; nicofuranose, which may be prepared as disclosed in Swiss Patent No. 366,523; nylidrin, which may be prepared as disclosed in U.S. Pat. Nos. 2,661,372 and 2,661,373; pentifylline, which may be prepared as disclosed above; pentoxifylline, which may be prepared as disclosed in U.S. Pat. No. 3,422,107; piribedil, which may be prepared as disclosed in U.S. Pat. No. 3,299,067; prostaglandin E₁, which may be prepared by any of the methods referenced in the Merck Index, Twelfth Edition, Budaveri, Ed., New Jersey, 1996, p. 1353; suloctidil, which may be prepared as disclosed in German Patent No. 2,334,404; tolazoline, which may be prepared as disclosed in U.S. Pat. No. 2,161,938; and xanthinol niacinate, which may be prepared as disclosed in German Patent No. 1,102,750 or Korbonits et al., Acta. Pharm. Hung., 1968, 38, 98. The disclosures of all such U.S. patents are incorporated herein by reference.

CCR5 antagonist can be used in combination with an anti-diabetic compound, i.e. any compound (e.g. insulin) used in the treating diabetes (especially Type II), insulin resistance, impaired glucose tolerance, or the like, or any of the diabetic complications such as neuropathy, nephropathy, retinopathy or cataracts. Additional examples of an anti-diabetic compound include, but are not limited to, a glycogen phosphorylase inhibitor, an aldose reductase inhibitor, a sorbitol dehydrogenase inhibitor, a glucosidase inhibitor, and an amylase inhibitor.

Any glycogen phosphorylase inhibitor known in the art that inhibits the bioconversion of glycogen to glucose-1-phosphate which is catalyzed by the enzyme glycogen phosphorylase may be used. Such glycogen phosphorylase inhibition activity may be readily determined according to standard assays (e.g., J. Med. Chem. 41 (1998) 2934-2938). A variety of glycogen phosphorylase inhibitors are known to those skilled in the art including those described in WO 96/39384 and WO 96/39385.

Any aldose reductase inhibitor known in the art that inhibits the bioconversion of glucose to sorbitol catalyzed by the enzyme aldose reductase. Aldose reductase inhibition may be readily determined according to standard assays (e.g., J. Malone, Diabetes, 29:861-864 (1980). “Red Cell Sorbitol, an Indicator of Diabetic Control”).

Any sorbitol dehydrogenase inhibitor known in the art that inhibits the bioconversion of sorbitol to fructose catalyzed by the enzyme sorbitol dehydrogenase may be used. Such sorbitol dehydrogenase inhibitor activity may be readily determined according to standard assays (e.g., Analyt. Biochem (2000) 280: 329-331). Examples of a suitable sorbitol dehydrogenase inhibitor include, but are not limited to, those described in U.S. Pat. Nos. 5,728,704 and 5,866,578.

Any glucosidase inhibitor known in the art that inhibits the enzymatic hydrolysis of complex carbohydrates by glycoside hydrolases, for example amylase or maltase, into bioavailable simple sugars, for example, glucose. Such glucosidase inhibition activity may be readily determined by those skilled in the art according to standard assays (e.g., Biochemistry (1969) δ: 4214).

A generally preferred glucosidase inhibitor includes an amylase inhibitor. Any amylase inhibitor known in the art that inhibits the enzymatic degradation of starch or glycogen into maltose may be used. Such amylase inhibition activity may be readily determined by those skilled in the art according to standard assays (e.g., Methods Enzymol. (1955) 1: 149).

Other preferred glucosidase inhibitors include, but are not limited to, acarbose and the various amino sugar derivatives related thereto (U.S. Pat. Nos. 4,062,950 and 4,174,439); adiposine (U.S. Pat. No. 4,254,256); voglibose, 3,4-dideoxy-4-[[2-hydroxy-1-(hydroxymethyl)ethyl]amino]-2-C-(hydroxymethyl-1)-D-epi-inositol, and the various N-substituted pseudo-aminosugars related thereto (U.S. Pat. No. 4,701,559); miglitol, (2R,3R,4R,5S)-1-(2-hydroxyethyl)-2-(hydro oxymethyl)-3,4,5-piperidinetriol, and the various 3,4,5-trihydroxypiperidines related thereto (U.S. Pat. No. 4,639,436); emiglitate, ethyl p-[2-[(2R,3R,4R,5S)-3,4,5-trihydroxy-2-(hydroxymethyl)piperidino]ethoxy]-benzoate, the various derivatives related thereto and pharmaceutically acceptable acid addition salts thereof (U.S. Pat. No. 5,192,772); MDL-25637, 2,6-dideoxy-7-O-.beta.-D-glucopyrano-syl-2,6-imino-D-glycero-L-gluco-heptitol, the various homodisaccharides related thereto and the pharmaceutically acceptable acid addition salts thereof (U.S. Pat. No. 4,634,765); camiglibose, methyl 6-deoxy-6-[(2R,3R,4R,5S)-3,4,5-trihydroxy-2-(hydroxymethyl)piperidino]-.alpha.-D-glucopyranoside sesquihydrate, the deoxynojirimycin derivatives related thereto, the various pharmaceutically acceptable salts thereof and synthetic methods for the preparation thereof (U.S. Pat. Nos. 5,157,116 and 5,504,078); pradimicin-Q; and salbostatin and the various pseudosaccharides related thereto (U.S. Pat. No. 5,091,524).

Any amylase inhibitor known in the art may be used. Examples include, but are not limited to, tendamistat and the various cyclic peptides related thereto (U.S. Pat. No. 4,451,455); A1-3688 and the various cyclic polypeptides related thereto (U.S. Pat. No. 4,623,714); and trestatin, consisting of a mixture of trestatin A, trestatin B and trestatin C and the various trehalose-containing aminosugars related thereto, (U.S. Pat. No. 4,273,765).

Additional examples of an anti-diabetic compound for use in a combination of the invention include: biguanides (e.g., metformin), insulin secretagogues (e.g., sulfonylureas and glinides), glitazones, non-glitazone PPAR.gamma. agonists, PPAR.beta. agonists, inhibitors of DPP-IV, inhibitors of PDE5, inhibitors of GSK-3, glucagon antagonists, inhibitors of f-1,6-BPase (Metabasis/Sankyo), GLP-1/analogs (AC 2993, also known as exendin-4), insulin and insulin mimetics (Merck natural products), PKC-beta inhibitors, and AGE breakers.

A compound of the invention can be used in combination with any anti-obesity agent known in the art. Anti-obesity activity may be readily determined according to standard assays known in the art. Examples of suitable anti-obesity agents include, but are not limited to, phenylpropanolamine, ephedrine, pseudoephedrine, phentermine, .beta.sub.3 adrenergic receptor agonists, apolipoprotein-B secretion/microsomal triglyceride transfer protein (apo-B/MTP) inhibitors, MCR-4 agonists, cholecystokinin-A (CCK-A) agonists, monoamine reuptake inhibitors (e.g., sibutramine—U.S. Pat. No. 4,929,629), sympathomimetic agents, serotoninergic agents, cannabinoid receptor antagonists (e.g., rimonabant (SR-141,716A)), dopamine agonists (e.g., bromocriptine—U.S. Pat. Nos. 3,752,814 and 3,752,888), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (e.g., tetrahydrolipstatin, i.e. orlistat), bombesin agonists, anorectic agents (e.g., a bombesin agonist), Neuropeptide-Y antagonists, thyroxine, thyromimetic agents, dehydroepiandrosterones or analogs thereof, glucocorticoid receptor agonists or antagonists, orexin receptor antagonists, urocortin binding protein antagonists, glucagon-like peptide-1 receptor agonists, ciliary neurotrophic factors (e.g., Axokine™), human agouti-related proteins (AGRP), ghrelln receptor antagonists, histamine 3 receptor antagonists or inverse agonists, neuromedin U receptor agonists, and the like.

Any thyromimetic agent known in the art may also be used in combination with a compound of the invention. Thyromimetic activity may be readily determined according to standard assays (e.g., Atherosclerosis (1996) 126: 53-63). Examples of suitable thyromimetic agents include, but are not limited to, those described in U.S. Pat. Nos. 4,766,121; 4,826,876; 4,910,305; 5,061,798; 5,284,971; 5,401,772; 5,654,468; and 5,569,674.

Any antihypertensive agent known in the art may be used in a combination of the invention. Antihypertensive activity may be determined according to standard tests (e.g. blood pressure measurements). Examples of suitable antihypertensive agents include, but are not limited to: (a) amlodipine and related dihydropyridine compounds as disclosed hereinabove; (b) calcium channel blockers as disclosed hereinabove; (c) angiotensin converting enzyme inhibitors (“ACE-Inhibitors”) as disclosed hereinabove; (d) angiotensin-II receptor antagonists as disclosed hereinabove; (e) beta-adrenergic receptor blockers as disclosed hereinabove; and (f) alpha-adrenergic receptor blockers (alpha- or α-blockers) as disclosed hereinabove, which may also be isolated from natural sources according to methods well known to those skilled in the art.

Any compound that is known to be useful in the treatment of Alzheimer's disease may be used in a combination of the invention. Such compounds include acetylcholine esterase inhibitors. Examples of known acetylcholine esterase inhibitors include, but not limited to donepezil (ARICEPT®; U.S. Pat. Nos. 4,895,841, 5,985,864, 6,140,321, 6,245,911 and 6,372,760), tacrine (COGNEX®; U.S. Pat. Nos. 4,631,286 and 4,816,456), rivastigmine (EXELON®; U.S. Pat. Nos. 4,948,807 and 5,602,17) and galantamine (REMINYL; U.S. Pat. Nos. 4,663,318 and 6,099,863).

In the above-described combinations, the CCR5 antagonist and additional therapeutic agent may be administered, in terms of dosage forms, either separately or in conjunction with each other; and in terms of their time of administration, either simultaneously or sequentially. Thus, the administration of one component agent may be prior to, concurrent with, or subsequent to the administration of the other component agent(s).

The present invention also relates to the pharmaceutically acceptable acid addition salts of compounds of the present invention. Pharmaceutically acceptable salts of the pharmaceutical agents listed herein include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, acid citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methanesulfonate, ethanesulfonate, benzenesulfonate, p-toluenesulfonate, methylsulphate, naphthylate, 2-napsylate, nicotinate, nitrate, orotate, oxalate, palmitate, pamoate (i.e., 1,1′-methylene-bis-(2-hydroxy-3-naphthoate)), phosphate/hydrogen phosphate/dihydrogen phosphate, acid phosphate, pyroglutamate, saccharate, stearate, succinate, sulfate, bisulfate, tannate, tartrate, bitartrate, tosylate, trifluoroacetate and xinofoate salts.

Suitable base salts are formed from bases which form non-toxic salts. Examples include the ammonium or water-soluble amine addition salts such as N-methylglucamine-(meglumine), and the lower alkanolammonium and other base salts of pharmaceutically acceptable organic amines, aluminium, arginine, benzathine, calcium, choline, diethylamine, diolamine, glycine, lysine, magnesium, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002), incorporated herein by reference.

The chemist of ordinary skill will recognize that certain compounds of this invention will contain one or more atoms which may be in a particular stereochemical or geometric configuration, giving rise to stereoisomers and configurational isomers. All such isomers and mixtures thereof are included in this invention. Hydrates and solvates of the compounds of this invention are also included.

Where the compounds of the present invention possess two or more stereogenic centers and the absolute or relative stereochemistry is given in the name, the designations R and S refer respectively to each stereogenic center in ascending numerical order (1, 2, 3, etc.) according to the conventional IUPAC number schemes for each molecule. Where the compounds of the present invention possess one or more stereogenic centers and no stereochemistry is given in the name or structure, it is understood that the name or structure is intended to encompass all forms of the compound, including the racemic form.

The compounds of this invention may contain olefin-like double bonds. When such bonds are present, the compounds of the invention exist as cis and trans configurations and as mixtures thereof. The term “cis” refers to the orientation of two substituents with reference to each other and the plane of the ring (either both “up” or both “down”). Analogously, the term “trans” refers to the orientation of two substituents with reference to each other and the plane of the ring (the substituents being on opposite sides of the ring).

This invention also includes isotopically-labeled compounds, which are identical to those described by formula I, except for the fact that one or more atoms are replaced by one or more atoms having specific atomic mass or mass numbers. Examples of isotopes that can be incorporated into compounds of the invention include isotopes of hydrogen, carbon, nitrogen, oxygen, sulfur, fluorine, and chlorine such as ²H, ³H, ¹³C, ¹⁴C, ¹⁵N., ¹⁸O, 17O, ¹⁸F, and ³⁶Cl respectively. Compounds of the present invention, prodrugs thereof, and pharmaceutically acceptable salts of the compounds or of the prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated (i.e., ³H), and carbon-14 (i.e., ¹⁴C), isotopes are particularly preferred for their ease of preparation and detectability. Further, substitution with heavier isotopes such as deuterium (i.e., ²H), can afford certain therapeutic advantages resulting from greater metabolic stability, for example increased in vivo half-life or reduced dosage requirements and, hence, may be preferred in some circumstances. Isotopically labeled compounds of this invention and prodrugs thereof can generally be prepared by carrying out the procedures disclosed in the schemes and/or in the Examples below, by substituting a readily available isotopically labelled reagent for a non-isotopically labeled reagent.

In this specification and in the claims that follow, reference will be made to a number of terms that shall be defined to have the following meanings:

As used herein in the specification, “a” or “an” may mean one or more. As used herein in the claim(s), when used in conjunction with the word “comprising”, the words “a” or “an” may mean one or more than one. As used herein “another” may mean at least a second or more.

“Compounds” or “compound” when used herein includes any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. By “tautomers” is meant chemical compounds that may exist in two or more forms of different structure (isomers) in equilibrium, the forms differing, usually, in the position of a hydrogen atom. Various types of tautomerism can occur, including keto-enol, ring-chain and ring-ring tautomerism. The expression “prodrug” refers to compounds that are drug precursors which following administration, release the drug in vivo via some chemical or physiological process (e.g., a prodrug on being brought to the physiological pH or through enzyme action is converted to the desired drug form). Exemplary prodrugs upon cleavage release the corresponding free acid, and such hydrolyzable ester-forming residues of the compounds of the present invention include but are not limited to those having a carboxyl moiety wherein the free hydrogen is replaced by (C₁-C₄)alkyl, (C₂-C₇)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

In one aspect, the compounds and combinations of this invention, their prodrugs and the salts of such compounds and prodrugs are all adapted to therapeutic use as agents that elevate HDL cholesterol activity in mammals, particularly humans. By virtue of their activity, these agents also reduce plasma levels of triglycerides, VLDL cholesterol, Apo-B, LDL cholesterol and their associated components in mammals, particularly humans. Moreover, these compounds and combinations are useful in equalizing LDL cholesterol and HDL cholesterol. Hence, these compounds and combinations are useful for the treatment and correction of the various dyslipidemias observed to be associated with the development and incidence of atherosclerosis and cardiovascular disease, including coronary artery disease, coronary heart disease, coronary vascular disease, peripheral vascular disease, hypoalphalipoproteinemia, hyperbetalipoproteinemia, hypertriglyceridemia, hypercholesterolemia, familial-hypercholesterolemia, low HDL and associated components, elevated LDL and associated components, elevated Lp(a), elevated small-dense LDL, elevated VLDL and associated components and post-prandial lipemia.

Given the negative correlation between the levels of HDL cholesterol and HDL associated lipoproteins, and the positive correlation between triglycerides, LDL cholesterol, and their associated apolipoproteins in blood with the development of cardiovascular, cerebral vascular and peripheral vascular diseases, the compounds and combinations of this invention, their prodrugs and the salts of such compounds and prodrugs, by virtue of their pharmacologic action, are useful for the prevention, arrestment and/or regression of atherosclerosis and its associated disease states. These include cardiovascular disorders (e.g., angina, ischemia, cardiac ischemia and myocardial infarction), complications due to cardiovascular disease therapies (e.g., reperfusion injury and angioplastic restenosis), hypertension, elevated cardiovascular risk associated with hypertension, stroke, atherosclerosis associated with organ transplantation, cerebrovascular disease, cognitive dysfunction (including, but not limited to, dementia secondary to atherosclerosis, transient cerebral ischemic attacks, neurodegeneration, neuronal deficient, and delayed onset or procession of Alzheimer's disease), elevated levels of oxidative stress, elevated levels of C-Reactive Protein, Metabolic Syndrome and elevated levels of HbA1C.

Because of the beneficial effects widely associated with elevated HDL levels, an agent which increases HDL cholesterol, also provides valuable avenues for therapy in a number of other disease areas as well.

Thus, given the ability of the compounds and combinations of this invention, their prodrugs and the salts of such compounds and prodrugs to alter lipoprotein composition, they are of use in the treatment of vascular complications associated with diabetes, lipoprotein abnormalities associated with diabetes and sexual dysfunction associated with diabetes and vascular disease. Hyperlipidemia is present in most subjects with diabetes mellitus (Howard, B. V. 1987. J. Lipid Res. 28, 613). Even in the presence of normal lipid levels, diabetic subjects experience a greater risk of cardiovascular disease (Kannel, W. B. and McGee, D. L. 1979. Diabetes Care 2, 120). It has been suggested that the abnormal increase in cholesterol transfer results in changes in lipoprotein composition, particularly for VLDL and LDL, that are more atherogenic (Bagdade, J. D., Wagner, J. D., Rudel, L. L., and Clarkson, T. B. 1995. J. Lipid Res. 36, 759). These changes would not necessarily be observed during routine lipid screening. Thus the present invention will be useful in reducing the risk of vascular complications as a result of the diabetic condition.

Agents that raise HDL cholesterol are useful in the treatment of inflammation due to Gram-negative sepsis and septic shock. For example, the systemic toxicity of Gram-negative sepsis is in large part due to endotoxin, a lipopolysaccharide (LPS) released from the outer surface of the bacteria, which causes an extensive inflammatory response. Lipopolysaccharide can form complexes with lipoproteins (Ulevitch, R. J., Johnston, A. R., and Weinstein, D. B., 1981. J. Clin. Invest. 67, 827-37). In vitro studies have demonstrated that binding of LPS to HDL substantially reduces the production and release of mediators of inflammation (Ulevitch, R. J., Johnston, A. R., 1978. J. Clin. Invest. 62, 1313-24). In vivo studies show that transgenic mice expressing human apo-Al and elevated HDL levels are protected from septic shock (Levine, D. M., Parker, T. S., Donnelly, T. M., Walsh, A. M., and Rubin, A. L. 1993. Proc. Natl, Acad. Sci. 90, 12040-44). Importantly, administration of reconstituted HDL to humans challenged with endotoxin resulted in a decreased inflammatory response (Pajkrt, D., Doran, W E., Koster, F., Lerch, P. G., Arnet, B., van der Poll, T., ten Cate, J. W., and van Deventer, S. J. H. 1996. J. Exp. Med. 184, 1601-08). The compounds and combinations of the present invention, by virtue of the fact that they raise HDL levels, attenuate the development of inflammation and septic shock. These compounds and combinations would also be useful in the treatment of endotoxemia, autoimmune diseases and other systemic disease indications, organ or tissue transplant rejection and cancer.

Generally, the compositions of this invention will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of CCR5 antagonists and combinations thereof and methods for their preparation will be readily apparent to those skilled in the art. Such compositions and methods for their preparation may be found, for example, in Remington's Pharmaceutical Sciences, 19th Edition (Mack Publishing Company, 1995), incorporated herein by reference.

Suitable modes of administration include oral, parenteral, topical, inhaled/intranasal, rectal/intravaginal, and ocular/aural administration.

The CCR5 antagonists and combinations thereof may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The CCR5 antagonists and combinations thereof may also be used in fast-dissolving, fast-disintegrating dosage forms such as those described in Expert Opinion in Therapeutic Patents, 11 (6), 981-986, by Liang and Chen (2001), incorporated herein by reference.

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. A preferred film coating is Opadry®.

The formulation of tablets is discussed in Pharmaceutical Dosage Forms: Tablets, Vol. 1, by H. Lieberman and L. Lachman (Marcel Dekker, New York, 1980), incorporated herein by reference.

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

CCR5 antagonists may be water-soluble or insoluble, A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864, incorporated herein by reference. Details of other suitable release technologies such as high energy dispersions and osmotic and coated particles are to be found in Pharmaceutical Technology On-line, 25(2), 1-14, by Verma et al (2001), incorporated herein by reference. The use of chewing gum to achieve controlled release is described in WO 00/35298, incorporated herein by reference.

The CCR5 antagonists and combinations thereof may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of the CCR5 antagonists used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

Formulations of CCR5 antagonsts also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999), incorporated herein by reference.

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The CCR5 antagonists and combinations thereof can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 μg to 10 mg of the compound of the invention. The overall daily dose will typically be in the range 1 μg to 200 mg which may be administered in a single dose or, more usually, as divided doses throughout the day.

Suitable formulations may also be produced by extemporaneous preparation in the pharmacy.

The CCR5 antagonists and combinations may be administered rectally or vaginally, for example, in the form of a suppository, pessary, vaginal ring, microbicide or enema. Cocoa butter is a traditional suppository base, but various alternatives may be used as appropriate.

Formulations for rectal/vaginal administration may be formulated to be immediate and/or modified release: Modified release formulations include delayed-, sustained-, pulsed-, controlled-, targeted and programmed release.

The CCR5 antagonists and combinations thereof may also be administered directly to the eye or ear, typically in the form of drops of a micronised suspension or solution in isotonic, pH-adjusted, sterile saline. Other formulations suitable for ocular and aural administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

CCR5 antagonists and combinations thereof may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148, incorporated herein by reference.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

Further aspects of the invention include the following.

A) Use of a CCR5 antagonist for elevating high density lipoprotein in a patient in need thereof.
B) Use of a synergistic combination of a CCR5 antagonist and an HMG-CoA reductase inhibitor in the preparation of a medicament for combined, separate or sequential administration for improving the lipid profile of a patient in need thereof.
C) Use of a synergistic combination of a CCR5 antagonist, an HMG-CoA reductase inhibitor and a CETP inhibitor in the preparation of a medicament for combined, separate or sequential administration for improving the lipid profile of a patient in need thereof.

All documents cited herein are hereby incorporated by reference.

The invention will now be described by reference to the accompanying examples. The examples presented below are intended to illustrate particular embodiments of the invention, and are not intended to limit the scope of the specification, including the claims, in any manner.

### Example 1

Treatment experienced HIV-1 patients infected with a CXCR4 using viral population were selected according to the following protocol and the first group with optimised background therapy (OBT) alone was compared against groups on OBT plus maraviroc once a day and OBT plus maraviroc twice a day.

### Selection Criteria

Patients Enrolled in the Trial:

(a) Were aged 16 or over;
(b) were infected with a dual/mixed CXCR4 using viral population as determined by Monogram Biosciences Phenosense™ (Trofile) HIV Entry assay (WO 02/099383; U.S. Pat. No. 5,837,464), or had an indeterminate viral tropism phenotype;
(c) had been on a stable antiviral regimen for at least 4 weeks prior to randomisation:
(d) had an HIV-1 RNA count of at least 5,000 copies/mL as measured by the Roche Amplicor HIV-1 Monitor (version 1.5)
(e) (i) had at least three months previous antiretroviral experience with at least one agent from three of the four antiretroviral drug classes: NTRIs, NNRTIs, protease inhibitors and fusion inhibitors (i.e. were triple class experienced); or
(ii) had documented resistance to at least one member of two of the four antiretroviral drug classes (i.e. were dual class resistant).

### Trial Treatments

The eligible patients were randomised into three groups based on the drug regimens they received.

Group 1: optimised background therapy (OBT) (3-6 antiretroviral drugs (not counting low dose ritonavir) of which at least one is active and no more than one is an NNRTI) plus placebo.

Group 2: optimised background therapy (as above) plus maraviroc 150 or 300 mg po taken once daily (QD).

Group 3: optimised background therapy (as above) plus maraviroc 150 or 300 mg po taken twice daily (BID).

Patients whose optimised background therapy did not contain a protease inhibitor (PI) or delavirdine (an NNRTI) were randomised to receive 300 mg doses of maraviroc once or twice daily (i.e. patients received a 300 mg equivalent dose of maraviroc QD or BID).

Patients taking a protease inhibitor were as follows: Group 1 (97%), Group 2 (91%) and Group 3 (88%).

Patients were stratified according to whether they had an HIV-1 RNA count of greater than or less than 100,000 copies/mL and were receiving enfuvirtide as part of their optimised background therapy. These patients were distributed evenly among the 3 patient groups.

The baseline HIV-1 RNA count was taken prior to the first dose (Day 1) and at multiple time points during the 24-week study period.

Prior to the first dose (Day 1), a fasting assessment was made, in which the following was measured and taken as the baseline measurement: total cholesterol, HDL, LDL, triglycerides, glucose and glycosolated haemoglobin.

HDL as part of a lipid profile was measured by Covance laboratories (Indianapolis, Ind.).

After 24 weeks of treatment or early termination, the same lipid parameters (in particular HDL levels) were measured again.

The results for HDL and LDL are shown in tables 1a to 3a.

Table 4a shows the mean viral load reduction after 24 weeks of treatment.

TABLE 1a
Population Change in LDL

| | Numbers of individuals with maximum % increase in | | | | | |
|---|---|---|---|---|---|---|
| Treatment | LDL cholesterol relative to baseline | | | | | |
| group | <0% | 0-5% | 5-<10% | 10-<20% | 20-<30% | ≧30% |
| Group 1 | 15 | 4 | 3 | 4 | 0 | 10 |
| N = 58 | | | | 14 | | |
| Group 2 | 11 | 2 | 5 | 9 | 4 | 7 |
| N = 63 | | | | 20 | | |
| Group 3 | 18 | 4 | 1 | 5 | 3 | 10 |

| | |
|---|---|
| N = 61 | 18 |

Data in Range 10 to >30 Within Sensitivity Range Of Assay

TABLE 2a
Population Change in HDL

| | Numbers of individuals with maximum % increase in | | | | | |
|---|---|---|---|---|---|---|
| Treatment | HDL cholesterol relative to baseline | | | | | |
| group | <0% | 0-5% | 5-<10% | 10-<20% | 20-<30% | ≧30% |
| Group 1 | 18 | 8 | 6 | 3 | 4 | 5 |
| N = 58 | | | | 12 | | |
| Group 2 | 17 | 6 | 4 | 7 | 2 | 9 |
| N = 63 | | | | 18 | | |
| Group 3 | 14 | 5 | 1 | 9 | 8 | 11 |

| | |
|---|---|
| N = 61 | 28 |

Data in Range 10 to >30 Within Sensitivity Range Of Assay

TABLE 3a
Mean % change in HDL

| | | Mean % difference from |
|---|---|---|
| Treatment | Mean % Change in | Group1 (placebo) (95% |
| group | HDL | Confidence Interval) |
| Group 1 | 2.3 | — |
| (n = 44) | | |
| Group 2 | 13.1 | 10.8 (−3.4, 25.0) |
| (n = 45) | | |
| Group 3 | 17.4 | 15.1 (1.1, 29.1) |
| (n = 48) | | |

TABLE 4a
Reduction in HIV-1 RNA viral load (VL)

| Variable | Group 1 | Group 2 | Group 3 |
|---|---|---|---|
| Mean VL reduction | −0.97 | −0.91 | −1.20 |
| (log₁₀ copies/mL) | | | |
| Treatment difference in VL | | +0.06 (−0.53, | −0.23 (−0.83, |
| (log₁₀ copies/mL) | | +0.64) | +0.36) |
| (95% confidence | | | |
| interval) | | | |

There is no increase over placebo (Group 1) in LDL levels after treatment with the maraviroc, as shown in Table 1a.

In Table 2a, however, the number of individuals with an increase in HDL-C levels above baseline, shows a clear relationship with increasing dose of maraviroc. The mean increase in HDL-C levels is summarised in table 3a. There was a clear dose-related increase in HDL-C levels following treatment with 300 mg BID maraviroc.

Since maraviroc is a CCR5 antagonist, it would not be expected to reduce the viral load (VL) in HIV-infected patients with a CXCR4 using viral population (a dual or mixed tropic viral population). As shown in Table 4a, the reduction in viral load after 24 weeks was similar for the Groups 1 and 2, and slightly but not statistically significantly greater for Group 3. Since there is no statistically significant reduction in viral load (VL) in this HIV patient group, the increase in HDL levels seems directly attributable to the treatment with the CCR5 antagonist, maraviroc.

### Example 2

### Background

The MERIT study was designed to compare the safety and efficacy of maraviroc (MVC) versus efavirenz (EFV), both administered with Combivir (CBV; a fixed-dose combination of zidovudine and lamivudine), in antiretroviral (ARV)-naive patients with R5HIV-1 by the Trofile™ assay (Monogram Biosciences, South San Francisco, Calif.). The MERIT study design included a fasting metabolic assessment at baseline and at Weeks 24 and 48 or at early study discontinuation, to evaluate the fasted lipid values in the MERIT study and their potential effect on cardiovascular risk.

### Methods

MERIT is a double-blind, randomized, multinational trial comparing the safety and efficacy of maraviroc 300 mg BID vs efavirenz 600 mg QD, both in combination with Combivir (zidovudine/lamivudine), in ARV-naive adult patients infected with only R5HIV by the Trofile® assay.
Patients experiencing toxicity to zidovudine or lamivudine were permitted to substitute an alternative NRTI.
A fasting lipoprotein profile was obtained for each patient at baseline, Week 24, and Week 48, or at early termination,
Median maximum changes in total cholesterol (TC), high-density lipoprotein (HDL) cholesterol, triglycerides (TG), calculated low-density lipoprotein (LDL) cholesterol, and total cholesterol/HDL ratio, as well as the proportion of patients in each treatment arm exceeding cutpoints identified in National Cholesterol Education Program (NCEP) ATP III clinical guidelines (NCEP Expert Panel on Detection, Evaluation, and Treatment of High Blood Cholesterol in Adults, JAMA 2001; 285:2486-2497), were compared between groups.
Overall cardiovascular disease risk was assessed for each patient using the Framingham equation (http://hp2010.nhibihin.net/atpiii/calculator.asp), which uses total cholesterol, HDL cholesterol; age, sex, smoking status, and use or nonuse of antihypertensive therapy (when systolic blood pressure is >120 mm/Hg) as variables to estimate 10-year absolute coronary heart disease (CHD) risk.
   Because data on smoking status were not collected in the study, CHD risk was compared between treatment groups according, to different smoking rate scenarios. For a 50% smoking rate scenario (which approximates to the smoking rate seen in the DAD Study-Friis-Møller N, et al. N Engl J Med 2003; 349:1993-2003), smoking status (yes/no) was randomly imputed to the study population 500 times, by sampling from a binomial distribution with a probability of success of 0.5. Smoking status was assumed to remain constant for the duration of the study.

### Results

A total of 721 patients at study centers in North America, Europe, South America, South Africa, and Australia were randomized and received at least one dose of study medication. Baseline characteristics were comparable between treatment groups (Table 1b).

TABLE 1b
Baseline characteristics*

| | EFV + CBV | | MVC + CBV | |
|---|---|---|---|---|
| | (N = 361) | | (N = 360) | |
| Age, years | 37 | (30, 43) | 35.5 | (30, 43) |
| Male, n (%) | 259 | (71.7) | 256 | (71.1) |
| Race, n (%) | | | | |
| White | 198 | (54.8) | 204 | (56.7) |
| Black | 133 | (36.8) | 123 | (34.2) |
| Asian | 5 | (1.4) | 6 | (1.7) |
| Other | 25 | (6.9) | 27 | (7.5) |
| Bodyweight, kg | 70.7 | (62.5, 80.1) | 72.3 | (62.6, 80.8) |
| Diabetes mellitus, n (%) | 6 | (1.7) | 5 | (1.4) |
| Supine systolic blood | 124 | (116, 131) | 123 | (114, 132) |
| pressure, mm/Hg | | | | |
| Supine diastolic blood | 76 | (70, 83) | 77 | (70, 84) |
| pressure, mm/Hg | | | | |
| HIV-1 RNA, log₁₀ copies/mL | 4.9 | (4.5, 5.2) | 4.9 | (4.4, 5.3) |
| CD4+ count, cells/mm³ | 259 | (189, 327) | 244 | (176, 320) |

*Values are median (lower quartile, upper quartile), unless otherwise stated

Baseline fasting lipid values were not significantly different between treatment groups (Table 2b). Approximately one in seven patients in each arm had a total cholesterol level 2200 mg/dL. A similar proportion of patients had a triglyceride level ≧200 mg/dL.
Five patients in the efavirenz arm and 11 patients in the maraviroc arm had LDL cholesterol values ≧160 mg/dL at baseline.
   This is the threshold at which the NCEP guidelines recommend considering LDL-lowering therapy in patients who have risk factors for CHD (e.g. hypertension, family history of premature CHD, older age, smoker) and a <10% 10-year risk for CHD.
Thirty-one patients in the efavirenz arm and 42 in the maraviroc arm had LDL cholesterol values 130 mg/dL at baseline.
   This is the threshold at which the NCEP guidelines recommend considering LDL-lowering therapy in patients who have ≧2 risk factors for CHD and a 10-20% 10-year risk for CHD.
Three patients in the efavirenz arm and five patients in the maraviroc arm were receiving LDL-lowering therapy at baseline. These patients, and patients who initiated LDL-lowering therapy during the study, were included in all the analyses presented here.

TABLE 2b
Baseline fasting lipid characteristics

| | | | Patients exceeding NCEP | |
|---|---|---|---|---|
| | | | lipid cutpoints n (%) (NCEP | |
| | Median | | Expert Panel on Detection | |
| | (lower quartile, upper | | EaTHBCiA. JAMA 2001; | |
| | quartile) | | 285: 2486-2497) | |
| | EFV + CBV | MVC + CBV | EFV + CBV | MVC + CBV |
| | (N = 361) | (N = 360) | (N = 361) | (N = 360) |
| Total cholesterol | 155 | 156 | 46 (12.8)^{†} | 50 (14.1)^{†} |
| (TC), mg/dL | (131, 178) | (131, 181) | | |
| LDL cholesterol, | 88 | 92 | 31 (8.9)^{†} | 42 (12.1)^{†} |
| mg/dL | (70, 111) | (71, 114) | 5 (1.4)^{‡} | 11 (3.2)^{‡} |
| HDL cholesterol, | 38 | 37 | N/A | N/A |
| mg/dL | (32, 46) | (31, 46) | | |
| TC:HDL | 4.0 | 4.2 | N/A | N/A |
| cholesterol | (3.2, 4.9) | (3.4, 5.0) | | |
| Triglycerides (TG), | 104 | 106 | 51 (14.2)^{‡} | 48 (13.5)^{‡} |
| mg/dL | (73, 162) | (76, 153) | | |

Baseline values were missing for up to 13 patients in each treatment group
^{†}Patients exceeding ‘borderline-high’ cutpoint (TC ≧200 mg/dL [≧5.2 mmol/L]; LDL ≧130 mg/dL [≧3.4 mmol/L])
^{‡}Patients exceeding ‘high’ cutpoint (LDL ≧160 mg/dL [≧4.1 mmol/L]; TG ≧200 mg/dL [≧2.3 mmol/L])

### On-Treatment NRTI Substitutions

A total of seven (1.9%) patients in the maraviroc arm and ten (2.9%) in the efavirenz arm changed their NRTIs from Combivir to Truvada (a fixed-dose combination of tenofovir and emtricitabine).

### On-Treatment Changes in Fasting Lipid Parameters

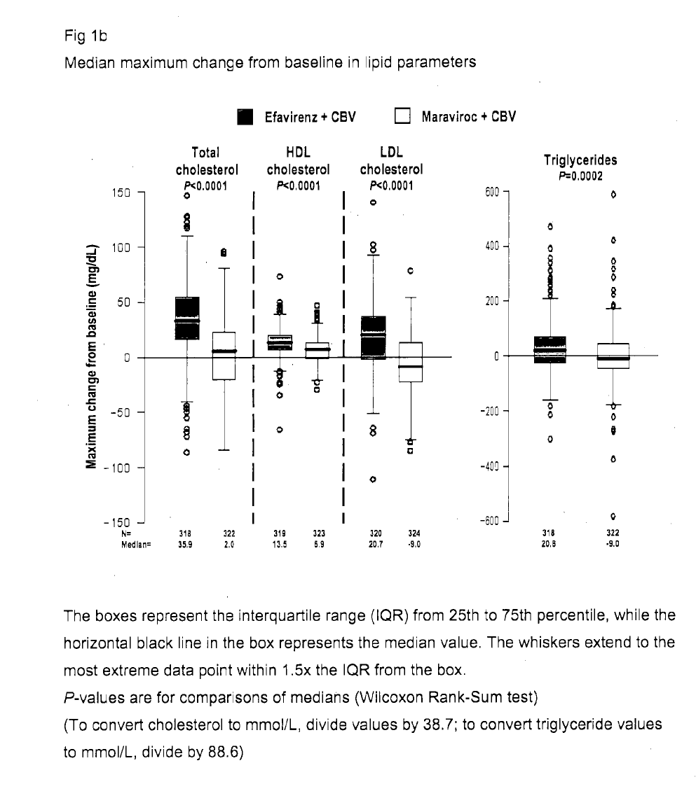

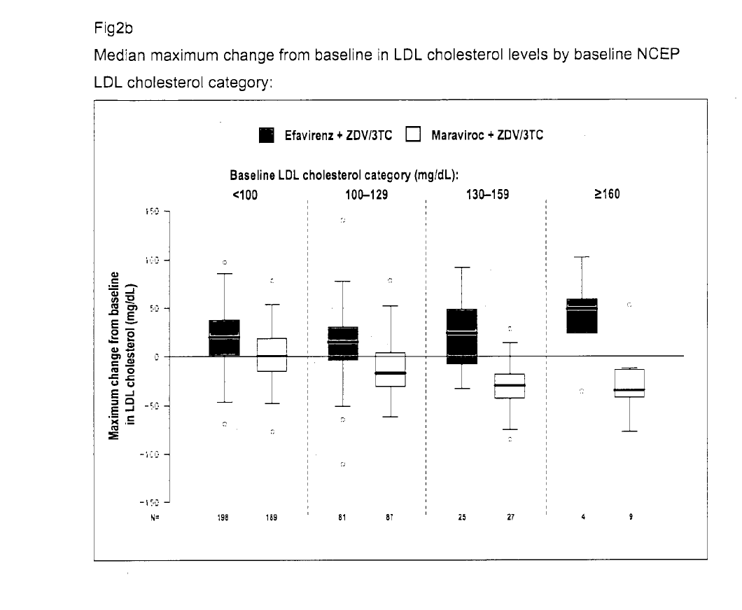

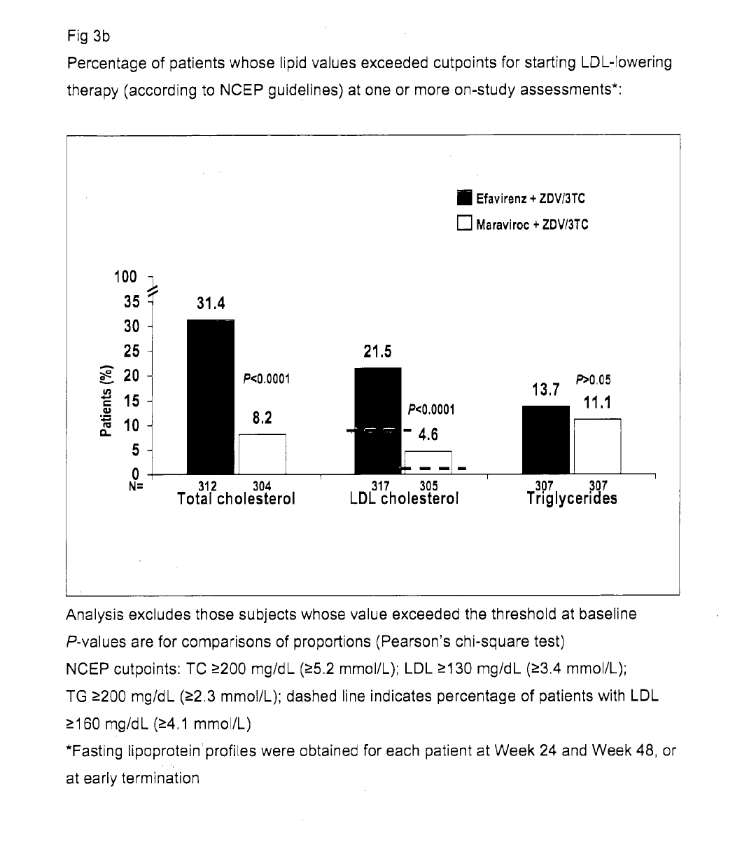

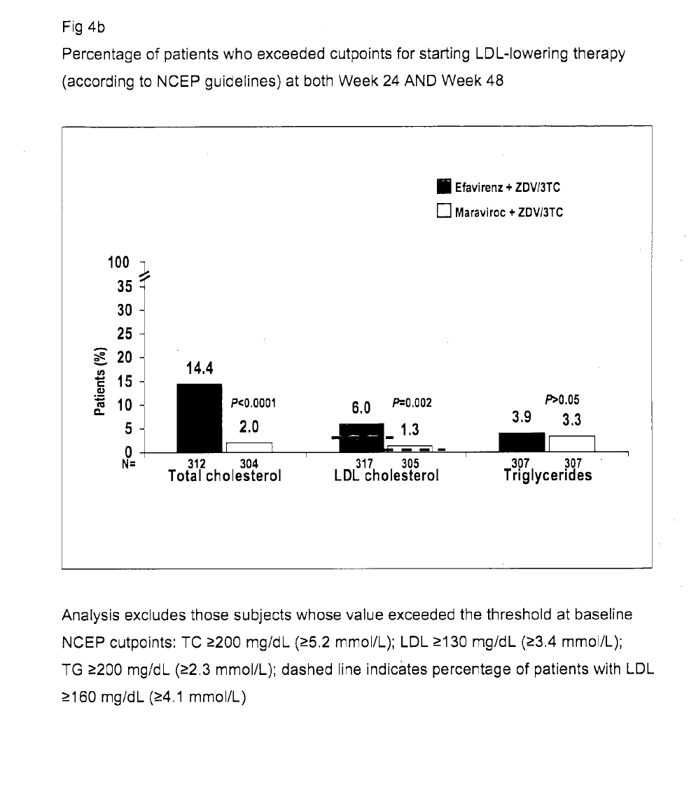

The median maximum changes from baseline in total cholesterol, HDL, cholesterol, LDL, cholesterol, and triglyceride levels were significantly greater in the efavirenz treatment group than in the maraviroc group (FIG. 1b).
The median decrease in the total cholesterol/HDL ratio was greater in the maraviroc group than in the efavirenz group (−0.54 vs −0.43, respectively; P=0.005).
While for efavirenz the change in LDL cholesterol was directly related to baseline LDL cholesterol, for maraviroc the change in LDL cholesterol was inversely related to baseline LDL cholesterol (FIG. 2b).
A greater percentage of patients receiving efavirenz therapy experienced increases in total cholesterol and LDL cholesterol levels at some point after baseline to ≧130 mg/dL, compared to those receiving maraviroc therapy (P<0.0001) (FIG. 3b).
   31/343 (9%) patients receiving efavirenz versus 3/336 (0.9%) patients receiving maraviroc developed LDL cholesterol levels during the study that were ≧160 mg/dL (P<0.0001).
Similarly, the proportions of patients that exceeded these cutpoints at both Weeks 24 and 48 were higher in the efavirenz group than in the maraviroc group (P<0,0001 for total cholesterol and P=0.002 for LDL cholesterol) (FIG. 4b).
   10/343 (2.9%) patients in the efavirenz arm versus 1/336 (0.3%) patients in the maraviroc arm had high on-treatment LDL cholesterol level (≧160 mg/dL) at both Week 24 and Week 48 (P=0.007).
These analyses did not include patients whose lipid levels already exceeded the thresholds at baseline.
A similar, low number of patients in each treatment group (six patients in the efavirenz arm and three patients in the maraviroc arm) initiated LDL-lowering therapy during the study.

### Cardiovascular Disease Risk

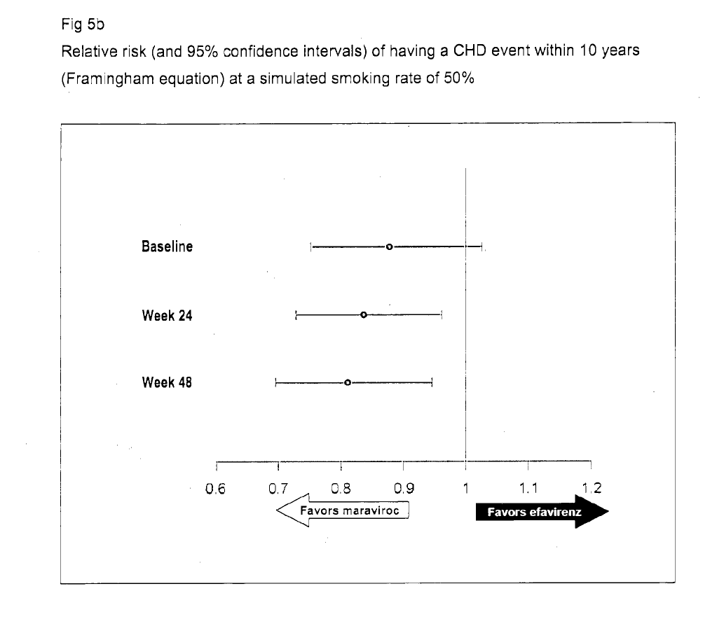

The relative risk of having a CHD event within 10 years for the two treatment groups, assuming a population smoking rate of 50% (see Methods), is shown in FIG. 5b. The risk was consistently higher in the efavirenz treatment group than in the maraviroc group at Week 24, and Week 48.
The absolute CHD 10-year risk (mean [SD]) in the maraviroc and efavirenz treatment groups was 2.1% (3.31%) and 3.0% (4.72%) at Week 24 and 2.2% (3.73%) and 3.3% (5.06%) at Week 48, respectively.

### Conclusions

At 48 weeks, increases from baseline in total cholesterol, LDL cholesterol, and triglycerides were significantly greater for patients receiving efavirenz+Combivir than for those receiving maraviroc+Combivir, with a higher proportion of efavirenz patients experiencing lipid levels above those recommended by NCEP guidelines. High LDL cholesterol levels and elevated triglycerides are both independent risk factors for cardiovascular disease (NCEP Expert Panel on Detection, Evaluation, and Treatment of High Blood Cholesterol in Adults JAMA 2001; 285:2486-2497).
   HDL cholesterol levels improved in both treatment groups. The median maximum increase was significantly greater in the efavirenz group than in the maraviroc group. The benefits of increasing HDL cholesterol levels independent of reductions in LDL cholesterol or triglyceride levels, in terms of reducing major cardiovascular outcomes, have not been proven (Singh I M, et al. JAMA 2007; 298:786-798).
There was a small but significant difference in total cholesterol/HDL ratios which decreased more from baseline in patients receiving maraviroc+Combivir than in those receiving efavirenz+Combivir.
The use of LDL-lowering therapy was infrequent and similar between the two treatment groups.
These data demonstrate that maraviroc has no negative impact on lipid profiles, and appears to have reduced the total cholesterol/HDL ratio. Overall, maraviroc is at least as lipid neutral as efavirenz and may offer some advantages compared to efavirenz, for example in those patients with elevated LDL cholesterol levels prior to treatment. Whether maraviroc will be associated with decreased progression of atherosclerosis in humans that is independent of lipid effects, as seen in the mouse model (Veillard N R, et al. Circ Res 2004; 94:253-261) remains to be determined.

### Example 3

Patients with cardiovascular disease and in need of lipid normalization treatments were selected and their lipid profiles and genotypes for variations in the CCR5 gene determined. Those with CCR5 variations known to lower CCR5 function had more favorable lipid profiles including higher HDL cholesterol and lower triglycerides.

### Selection Criteria

Patients were enrolled in either the TNT (1) or IDEAL (2) trial for assessment of response to various statins and impact on cardiovascular disease. Both trials were longitudinal in design and followed patient responses to statins over a period of years but we used only the initial lipid values determined at the screening visits prior to randomization.

### In TNT, Patients Were:

aged 35-75
found to have LDL cholesterol levels between 130 and 250 mg/dl at screening
found to have less than 130 mg/dl LDL cholesterol after 8 weeks of treatment with atorvastatin
identified with clinically evident coronary heart disease

### In IDEAL, Patients Were:

less than 80 years old
identified with coronary heart disease
eligible for statin treatment

### Genetics

CCR5 is a 6058 base pair gene located on chromosome 3p21.31 spanning positions 46,386,636 to 46,392,694. It resides within a chemokine receptor gene cluster on chromosome 3 and lies approximately 9 kb 3′ of the CCR2 gene and 31 kb 5′ of the CCRL2 gene. CCR5 consists of 3 exons and produces a 7 transmembrane G-protein coupled receptor protein. The del32 polymorphism has been shown to inactivate the CCR5 gene product (3) so it was the focus of our analysis though some other polymorphisms in or near CCR5 showed similar results. In both trials, blood was collected and DNA prepared from all patients who provided appropriate informed consent. This DNA was then genotyped using either Taq Man® or SNPlex™ technologies according to the manufacturer's (ABI) instructions (4). Only patients who self-identified as being of European ancestry were included in the analysis because the polymorphism was known to be present at much higher frequency in this group than in the other available populations. 5656 individuals were successfully genotyped for del32 in the TNT cohort with a call rate of about 99%. This population was in Hardy-Weinberg equilibrium (p=0.1). In the IDEAL trial, 6555 were successfully genotyped with a call rate of greater than 99%. This population was also in Hardy-Weinberg equilibrium (p=0.61).

### Analysis

Fasting lipid levels were determined according to standard procedures as described (1, 2). A linear regression analysis of genotype with the log transformed values of either HDL cholesterol or triglycerides was performed using age and gender as co-variates. P values were generated for each phenotype for each trial. In TNT, del32 is associated with higher HDL cholesterol only in the recessive mode meaning that individuals who have two copies of the deletion have higher HDL-C levels than those with either one or two copies or the common allele. If not corrected for multiple testing, this association is significant with p=0.007. The deletion is associated with lower triglycerides with an uncorrected p value of 0.007. No significant association was found with LDL-C. In IDEAL, the deletion allele was associated with higher HDL cholesterol with p=0.019 when analyzed in an additive manner. In IDEAL, the deletion allele was associated with lower triglycerides was associated with p=0.029 when analyzed in an additive manner.
Thus, genetic data is consistent with the hypothesis that reducing the function of CCR5, in this case through the means of a genetic variant but more generally through therapeutic means, should have a beneficial impact on lipid profiles by raising HDL-C and lowering triglycerides with little or no impact on LDL cholesterol levels.

TABLE 1c
Lipid levels for the three del32 genotypes and
minor allele frequencies

| Trial | Lipid | Number | MAF | Ins/Ins | Ins/Del | Del/Del |
|---|---|---|---|---|---|---|
| TNT | HDL | 5656 | 0.110 | 48.3 | 48.5 | 52.0 |
| TNT | TG | 5656 | 0.110 | 203.5 | 202.8 | 170.6 |
| IDEAL | HDL | 6555 | 0.117 | 45.8 | 46.6 | 47.5 |
| IDEAL | TG | 6555 | 0.117 | 149.4 | 145.0 | 143.5 |

Abbreviations:
Del, Deletion allele;
Ins, Insertion allele;
HDL, high density lipoprotein;
LDL, low density lipoprotein;
MAF, minor allele frequency;
TG, triglycerides

1. LaRosa, J. C. et al. Intensive lipid lowering with atorvastatin in patients with stable coronary disease, N Engl J Med 352, 1425-35 (2005).
2. Pedersen et al. High-Dose Atorvastatin vs Usual-Dose Simvastatin for Secondary Prevention After Myocardial Infarction. The IDEAL Study: A Randomized Controlled Trial. JAMA 294:2437-2445 (2005).
3. P A Zimmerman et al. Inherited resistance to HIV-1 conferred by an inactivating mutation in CC chemokine receptor 5. Mol. Med. 3: 23-36 (1997).
4. F M De La Vega, K D Lazaruk, M D Rhodes, and M H Wenz, Assessment of two flexible and compatible SNP genotyping platforms: TabMan® SNP genotyping assays and SNPlex™ genotyping system. Mutation Research 573: 111-135 (2005).
   Throughout this application, various publications are referenced. The disclosures of these publications in their entireties are hereby Incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. Use of a CCR5 antagonist compound for the preparation of a medicament for elevating high density lipoprotein (HDL) particles in a patient.

2. Use of a CCR5 antagonist compound for the preparation of a medicament for improving plasma lipid profile in a patient.

3. Use of a CCR5 antagonist compound for the preparation of a medicament for reducing triglycerides in a patient.

4. Use of claim 1 wherein the patient is infected with HIV.

5. Use as claimed in claim 4 wherein the patient is infected with a CXCR4 virus using HIV viral population.

6. Use as claimed in claim 5 wherein the viral population of the HIV patient contains more than 50% CXCR4 virus.

7. Use claim 1 wherein the CCR5 antagonist compound is selected from maraviroc, vicriviroc, NCB-9471, PRO-140, CCR5 mAb004, 8-[4-(2-butoxyethoxy)phenyl]-1-isobutyl-N-[4-[[(1-propyl-1H-imadazol-5-yl)methyl]sulphinyl]phenyl]-1,2,3,4-tetrahydro-1-benzacocine-5-carboxamide, methyl1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carboxylate, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, and N-{(1S)-3-[3-endo-(5-isobutyryl-2-methyl-4,5,6,7-tetrahydro-1H-imidazo[4,5-c]pyridin-1-yl)-8-azabicyclo[3.2.1]oct-8-yl]-1-(3-fluorophenyl)propyl}acetamide) or a pharmaceutically acceptable salt thereof.

8. Use as claimed in claim 7 wherein the CCR5 antagonist compound is maraviroc or a pharmaceutically acceptable salt thereof.

9. Use of claim 4 wherein the HIV patient is taking at least a protease inhibitor compound or a nucleoside or nucleotide reverse transcriptase inhibitor compound.

10. Use of claim 1 wherein the patient is diagnosed with a disease which is affected by low levels of HDL cholesterol and/or high levels of LDL-cholesterol and triglycerides, wherein the disease is selected from atherosclerosis, plaque formation, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia, myocardial infarction, metabolic syndrome, obesity and diabetes.

11. A CCR5 antagonist compound for use in elevating high density lipoprotein (HDL) particles in a patient.

12. A CCR5 antagonist compound for use in improving the plasma lipid profile in a patient.

13. A CCR5 antagonist compound for use in reducing triglycerides in a patient.

14. A CCR5 antagonist compound for use as claimed in claim 11, wherein the patient is infected as described in claim 4.

15. A CCR5 antagonist compound for use as claimed in claim 11, wherein the CCR5 antagonist compound is selected from maraviroc, vicriviroc, NCB-9471, PRO-140, CCR5 mAb004, 8-[4-(2-butoxyethoxy)phenyl]-1-isobutyl-N-[4-[[(1-propyl-1H-imadazol-5-yl)methyl]sulphinyl]phenyl]-1,2,3,4-tetrahydro-1-benzacocine-5-carboxamide, methyl1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carboxylate, ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate, and N-{(1S)-3-[3-endo-(5-isobutyryl-2-methyl-4,5,6,7-tetrahydro-1H-imidazo[4,5-c]pyridin-1-yl)-8-azabicyclo[3.2.1]oct-8-yl]-1-(3-fluorophenyl)propyl}acetamide) or a pharmaceutically acceptable salt thereof.

16. A CCR5 antagonist compound for use as claimed in claim 15, wherein the CCR5 antagonist compound is maraviroc or a pharmaceutically acceptable salt thereof.

17. A CCR5 antagonist compound for use as claimed in claim 14, 15 or 16, wherein the HIV patient is taking at least a protease inhibitor compound or a nucleoside or nucleotide reverse transcriptase inhibitor compound.

18. A pharmaceutical composition comprising:
(i) a CCR5 antagonist compound;
(ii) an HMG-CoA reductase inhibitor compound; and
(iii) a pharmaceutically acceptable carrier.

19. The composition as claimed in claim 18 wherein the HMG-CoA reductase inhibitor compound is atorvastatin or a pharmaceutically acceptable salt thereof.

20. The composition as claimed in claim 18 or claim 19 wherein the CCR5 antagonist compound is maraviroc or a pharmaceutically acceptable salt thereof.

21. The composition of claim 18 which further comprises a cholesteryl ester transfer protein (CETP) inhibitor compound or a pharmaceutically acceptable salt thereof.

22. A pharmaceutical composition comprising:
(i) a CCR5 antagonist compound;
(ii) a cholesteryl ester transfer protein (CETP) inhibitor compound; and
(iii) a pharmaceutically acceptable carrier

23. The composition of claim 22 wherein the CCR5 antagonist compound is maraviroc or a pharmaceutically acceptable salt thereof.

24. The composition of claim 21, wherein the cholesteryl ester transfer protein (CETP) inhibitor compound is cis-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}cyclohexyl)-acetamide; or (2R)-3-{[3-(4-Chloro-3-ethyl-phenoxy)-phenyl]-[[3-(1,1,2,2-tetrafluoro-ethoxy)-phenyl]-methyl]-amino}-1,1,1-trifluoro-2-propanol or a pharmaceutically acceptable salt thereof.

25. A pharmaceutical composition of claim 18, for the treatment of a disease selected from atherosclerosis, plaque formation, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia, myocardial infarction, metabolic syndrome, obesity and diabetes.
